(19) United States
(12) Patent Application Publication
(10) Pub. No.: US 2013/0004946 A1
(43) Pub. Date: Jan. 3, 2013

(54) Compositions and Methods for Genetic Manipulation and Monitoring of Cell Lines

(75) Inventors: Jonathan Chesnut, Carlsbad, CA (US); Bhaskar Thyagarajan, Carlsbad, CA (US); Antje Taliana, Carlsbad, CA (US); Pauline Lieu, San Diego, CA (US); Mahendra Rao, Timonium, MD (US); Robert Bennett, Encinitas, CA (US); Robert Burrier, Verona, WI (US); Uma Lakshmipathy, Carlsbad, CA (US); Ying Liu, San Marcos, CA (US)

(73) Assignee: Life Technologies Corporation, Carlsbad, CA (US)

(21) Appl. No.: 13/448,290

(22) Filed: Apr. 16, 2012

Related U.S. Application Data
(63) Continuation of application No. 12/016,415, filed on Jan. 18, 2008, now abandoned.
(60) Provisional application No. 60/885,843, filed on Jan. 19, 2007.
(60) Provisional application No. 60/969,051, filed on Aug. 30, 2007.

(51) Int. Cl. C12N 15/90; C12Q 1/68

(52) U.S. Cl. 435/6.1; 435/462; 435/468; 435/471

(57) ABSTRACT

The disclosure relates generally to stem cell biology and more specifically to genetic manipulation of stem cells. Methods and compositions using recombinational cloning techniques are disclosed which allow the construction and insertion of complex genetic constructs into embryonic and adult stem cells and progenitor cells. The methods disclosed will allow the harvesting of adult stem cells pre-engineered with integration sites to facilitate early passage genetic modification.

This application is a continuation of application Ser. No. 12/016,415, filed Jan. 18, 2008, which claims the benefit under 35 U.S.C. §119(e) of Provisional Application Ser. Nos. 60/885,843 filed on Jan. 19, 2007, and 60/969,051, filed Aug. 30, 2007, the disclosures of which are hereby incorporated in their entireties by reference.

### BACKGROUND OF THE INVENTION

1. Field of the Invention

The invention relates generally to cell biology and more specifically to genetic manipulation of cells such as stem cells. Methods and compositions using recombinational cloning techniques are disclosed which allow the construction and insertion of nucleic acid molecules, for example complex genetic constructs, into cells such as embryonic stem cells, adult stem cells and progenitor cells. Methods disclosed will allow, in part, for the harvesting of adult stem cells pre-engineered with integration sites to facilitate early passage genetic modification.

2. Background Information

Methods of inserting heterologous gene expression constructs into mammalian cells such as electroporation, lipid-based transfection, and viral gene transfer have proven useful but often result in variable expression levels due to lack of control of plasmid copy number or site of integration. Upon selection of stable tranfectants, variable copy number and random genomic insertion often result in differences in expression levels when comparing multiple cell clones. These problems are especially onerous in stem cell systems since chromosomal remodeling and locus silencing (which occurs during differentiation) leads to inhibition of expression from some clones (termed clonal variegation).

The limited ability of adult stem cells to proliferate in culture poses a challenge for standard gene expression studies since stable transfection and clonal isolation are required for efficient, controllable expression in cells. To create a clonal population of stably transfected cells, the single transfected cells must be isolated and propagated through at least 20 doublings in order to obtain a usable pool. In contrast to immortal cell lines which can proliferate in culture indefinitely, it has been known for some time that mortal (adult stem and progenitor) cells proliferate in culture for approximately 30-35 population doublings at which point they continue metabolizing but cease to divide. This so-called Hayflick limit is thought to be a result of, among other things, progressive shortening of their chromosomes during each round of DNA replication. After multiple rounds of replication the cells finally reach a point where as yet undefined genes critical for proliferation are disrupted or inactivated. Cellular senescence is a clear limitation for both human and mouse adult stem cell research.

### SUMMARY OF THE INVENTION

The present invention provides methods and compositions which allow, in part, for the introduction of nucleic acids into cells. In some embodiments, the cells are stem cells. Cells used in the invention may be embryonic stem cells, adult stem cells or progenitor cells. In some embodiments the introduced nucleic acids are pre-existing genetic constructs while in other embodiments, disclosed methods allow for rapid assembly of complex genetic constructs. The present invention also allows for harvesting of cells (e.g., stem cells) pre-engineered with integration sites to facilitate early passage genetic modification. In some embodiments, harvested cells are stem cells and in further embodiments cells are harvested from an animal, for example, a rodent such as a mouse. The invention makes use, in part, of site-specific recombination sites inserted into the genomes of cells. In some aspects, the inserted recombination sites allow for targeted insertion of nucleic acid molecules, for example complex genetic constructs, into the genome of the cell.

Some aspects of the invention employ recombinational cloning techniques. These techniques involve, but are not limited to, homologous recombination and site specific recombination. A non-limiting example of site specific recombination is the GATEWAY™ system (Invitrogen Corp. Carlsbad, Calif.; Gateway™ Technology Manual, Version E Catalog Nos. 12535-019 and 12535-027, Sep. 22, 2003). Techniques such as these may be used to assemble complex expression vectors for insertion into cells. The integration of nucleic acids which can be constructed by such techniques can be directed to particular locus within the genome of cells by inserting one or more recombination sites such as wild type recombination sites at one or more (e.g., two, three, four, five, seven, ten etc.) loci in the genome. Criteria for selecting genomic loci for insertion of recombination sites include, but are not limited to, proximity to highly active promoters and regions of the chromosome that are known to be highly expressed (e.g. open chromatin). These recombination sites can then be used as targets for nucleic acids (e.g. plasmids, vectors, gene cassettes etc.) which are engineered to have complimentary recombination sites. The insertion of these nucleic acids into cells allows for the generation of cells which may be used in any number of ways. For example, cells generated by methods disclosed herein may be used in studies on the effects of drug compounds on cellular differentiation, protein-protein interactions and cell specific signaling pathways in the context of a normal cellular environment.

Among the possible embodiments of the invention are two embodiments outlined in FIGS. 1 and 2. The genetic tool box typically comprises nucleic acid molecules having one or more recognition sites (e.g., two, three, four, five, seven, ten, etc. recombination sites, restriction sites, and/or topoisomerases sites). Recognition sites allow for manipulation of elements of the genetic tool box in a determinable fashion without loss of an essential biological function. When present, recombination sites may function in homologous recombination or in site specific recombination reactions. In some embodiments the recognition sites are located at the ends of the nucleic acid molecule. Nucleic acids of the tool box may further comprise one or more selectable markers (e.g., two, three, four, five, seven, ten, etc.). Nucleic acids used in the invention may be single or double stranded and may be DNA or RNA. Further, nucleic acid molecules may encode for a protein or peptide or may encode a nucleic acid molecule such as an RNAi molecule. In some embodiments the genetic tool box may comprise entry clones as those used in the GATEWAY™ recombination system.

An expression vector refers to a nucleic acid molecule (preferably DNA) that provides a useful biological or biochemical property to an insert such as a nucleic acid molecule from the Genetic Tool Box. A vector may be a nucleic acid molecule comprising all or a portion of a viral genome. Examples include plasmids, phages, autonomously replicating sequences (ARS), centromeres, and other sequences that are able to replicate or be replicated in vitro or in a host cell, or to convey a desired nucleic acid segment to a desired location within a host cell. An expression vector can have one or more recognition sites (e.g., two, three, four, five, seven, ten, twelve, fifteen, twenty, thirty, fifty, seventy-five, one hundred, two hundred, etc. recombination sites, restriction sites, and/or topoisomerases sites) these recognition sites can often be used to manipulate, in a determinable fashion and without loss of an essential biological function of the expression vector, the insertion of nucleic acid fragments in order to bring about their expression. Expression vectors can further provide primer sites (e.g., for PCR), transcriptional and/or translational initiation and/or regulation sites, recombinational signals, replicons, selectable markers, etc.

Clearly, methods of inserting a desired nucleic acid fragment that do not require the use of recombination, transpositions or restriction enzymes (such as, but not limited to, uracil N-glycosylase (UDG) cloning of PCR fragments (U.S. Pat. Nos. 5,334,575 and 5,888,795, both of which are entirely incorporated herein by reference), TA cloning, and the like) can also be applied to clone a fragment into an expression vector to be used according to the present invention. An expression vector can further contain one or more selectable markers (e.g., two, three, four, five, seven, ten, etc.) suitable for use in the identification of cells transformed with the expression vector.

An embryonic or adult stem cell refers to an unspecialized cell capable of developing into a variety of specialized cells and tissues. Embryonic stem cells are found in very early embryos and are derived from a group of cells called the inner cell mass, a part of a blastocyst. Embryonic stem cells are self-renewing and can form all cell types found in the body (pluripotent). Adult stem cells may be obtained from, among other sources, blood, bone marrow, brain, pancreas, amniotic fluid and fat of adult bodies. Adult stem cells may renew themselves and differentiate to give rise to all the specialized cell types of the tissue from which it originated and potentially cell types associated with other tissues (multipotent).

The “Target Site” shown in FIGS. 1 and 2 refers to a recognition site that may be inserted into the genome of a cell, such as a stem cell. Target sites may be a recombination site, a restriction site and/or a topoisomerase site. One or more recognition sites (e.g., two, three, four, five, seven, ten, etc.) may be inserted into the genome of the stem cell. Target sites may be inserted at specific locations within the genome of the stem cell. In embodiments where multiple target sites are inserted, the specificity of each site may be different, allowing for insertion of nucleic acids at specific locations in the genome.

In some embodiments, target sites may be present on additional genetic material present in the cell, for example artificial chromosomes. Examples of such additional genetic material include, but are not limited to, the artificial chromosomes described in U.S. Pat. Nos. 6,025,155, 6,077,697 and 6,743,967 which are incorporated herein by reference in their entirety.

As outlined in FIG. 1, for some embodiments of the invention employing embryonic stem cells (ESC), there is considerable flexibility in how the invention may be applied. In some embodiments, one or more target recognition sites (e.g., two, three, four, five, seven, ten, etc.) may be inserted into an ESC and then the ESC may be used to produce a transgenic animal such as a transgenic mouse (or Platform Mouse). Although a mouse is used in these examples, the invention is not limited to using a mouse. The invention is applicable to any animal. In many instances, cells in a Platform Mouse will have a target recognition site present in its' genome. Methods described herein allow for the efficient insertion of nucleic acids into both embryonic and adult stem cells derived from a Platform Mouse or other animal. This allows for recovery of genetically modified stem cells at a low passage number without the need for lengthy cloning procedures.

In other embodiments, the target recognition site is inserted into the ESC and then the nucleic acid may be inserted into the genome of the targeted ESC. This genetically modified ESC may then be used to derive a transgenic animal wherein the cells of the transgenic animal contain the genetic modification, i.e. an engineered transgenic mouse (see FIG. 1). Alternatively, the genetically modified ESC can be used directly without additional modification.

Some embodiments employing adult stem cells are outlined in FIG. 2. In such embodiments, one or more target recognition sites (e.g., two, three, four, five, seven, ten, etc.) may be inserted into the adult stem cell. The adult stem cells may then be genetically modified by inserting a nucleic acid molecule. Such methods allow, in part, the efficient isolation of modified cells at low passage number without the need for lengthy cloning procedures. In further embodiments, the modified cells are stem cells.

FIG. 3 is a schematic which illustrates how the invention may be used to control differentiation of genetically engineered cells. In embodiments depicted in FIG. 3, nucleic acids may have two recombination sites (R1, R2 etc.) flanking a selectable marker. The selectable marker may be either positive (Pos) or negative (Neg) and may not be under the control of a promoter. Common selectable markers include those for resistance to antibiotics such as ampicillin, tetracycline, kanamycin, bleomycin, streptomycin, hygromycin, neomycin, Zeocin™, and the like. Selectable auxotrophic genes include, for example, hisD, that allows growth in histidine free media in the presence of histidinol. Selectable markers also include fluorescent proteins and membrane tags such as pHOOK which may be used with magnetic beads, cell sorters or other means to separate cells. The selectable marker may also encode a regulatory molecule such as an RNAi molecule which controls the expression of a critical gene.

One or more (e.g., two, three, four, five, seven, ten, twelve, fifteen, twenty, thirty, fifty, seventy-five, one hundred, two hundred, etc.) of the nucleic acid molecules depicted in FIG. 3 may be transfected into a cell such as a stem cell where they may become integrated into the chromosome by a recombination reaction. The number of nucleic acid molecules which may be integrated into the genome is limited only by the number of unique recombination sites available. Individual nucleic acid molecules may be linked together in intermediate molecules which are then transfected into the cell.

In some embodiments, recombination sites in the genome of the cell are located adjacent to developmentally related promoters (P1, P2 etc.). Activity of a developmentally related promoter may be limited to a specific stage of development, a certain lineage or type of cell or to a particular differentiation state. When a nucleic acid molecule becomes integrated adjacent to such a promoter, the selectable marker in the nucleic acid molecule falls under the control of the promoter. Because the activity of the developmentally related promoter may be linked to a differentiation state, cell lineage or cell type, the activity of the selectable marker may also become linked to a differentiation state, cell lineage or cell type. Therefore, as the cell begins to differentiate, selection can be applied to select for or against cells following a particular differentiation pathway. For example, if the P1 promoter is associated with a differentiation pathway or cell type that is not desired, negative selection can be applied eliminating cells which follow the non-desired pathway. Alternatively, if the P2 promoter is associated with a desired differentiation pathway or cell type then positive selection may be applied to enrich for cells following the desired pathway. In a further example, nucleic acid molecules as depicted in FIG. 3 may be transfected into a mixed population of cells. When activity of developmentally related promoters in each of the cell types present in the mixed population is known, appropriate selection may be applied to select a single cell type from the mixed population.

Examples of suitable developmentally related promoters include the Oct-4 promoter. In addition, promoters which are cell-type-specific, stage-specific, or tissue-specific can be used. For example, several liver-specific promoters, such as the albumin promoter/enhancer, have been described (see, e.g., Shen et al., 1989, DNA 8:101-108; Tan et al., 1991, Dev. Biol. 146:24-37; McGrane et al., 1992, TIBS 17:40-44; Jones et al., J. Biol. Chem. 265:14684-14690; and Shimada et al., 1991, FEBS Letters 279:198-200). Where promoters active in liver are desired, an α-fetoprotein promoter is particularly useful. This promoter is normally active only in fetal tissue; however, it is also active in liver tumor cells (Huber et al., 1991, Proc. Natl. Acad. Sci. 88:8039-8043). Further examples include α-1-antitrypsin, pyruvate kinase, phosphenol pyruvate carboxykinase, transferrin, transthyretin, α-fetoprotein, α-fibrinogen, or β-fibrinogen. An albumin promoter may be used. Other liver-specific promoters include promoters of the genes encoding the low density lipoprotein receptor, α 2-macroglobulin, α 1-antichymotrypsin, α 2-HS glycoprotein, haptoglobin, ceruloplasmin, plasminogen, complement proteins (C1q, C1r, C2, C3, C4, C5, C6, C8, C9, complement Factor I and Factor H), C3 complement activator, 3-lipoprotein, and α1-acid glycoprotein. Additional tissue-specific promoters may be found in the Tissue-Specific Promoter Database, TiProp (Nucleic Acids Research, 34:D104-D107 (2006)).

In some embodiments, the present invention comprises a method for inserting genetic material into cells by transfecting the cells with a nucleic acid such as a plasmid. In some instances the plasmid further comprises one or more of the following: a first recombination site, a first selectable marker and a second selectable marker. In specific embodiments, the first selectable marker may be used to select cells in which the nucleic acid has been integrated into the genome. The selected cells with the integrated nucleic acid may then be transfected with a second nucleic acid. The second nucleic acid may further comprise one or more of the following: a genetic element for expression in the cell, a promoter and a second recombination site. The selected cells may further be provided with a recombinase specific for the first and second recombination sites such that the second nucleic acid may be inserted into the genome of the cell. In some embodiments the insertion is accomplished by site-specific recombination. In certain embodiments, the second conditional selectable marker may not be operably linked to a genetic control element such as a promoter and so may not be expressed. In such embodiments, a promoter in the second nucleic acid may be positioned so that when the second nucleic acid is inserted into the genome it becomes operably linked to the second conditional selectable marker so that cells with the integrated second nucleic acid may be selected using the second selectable marker. The invention further includes compositions used in the above methods as well as cells produced by these methods.

In some embodiments, cells receiving genetic material are prokaryotic or eukaryotic cells. In specific embodiments, cells may be a stem cell or progenitor cell. When stem cells are used in the practice of the invention, the stem cells may be multipotent adult stem cells or pluripotent embryonic stem cells.

The insertion of nucleic acid into cells may be random or specifically targeted. The invention is not limited by the mechanism of how the nucleic acid is inserted into the genome but possible mechanisms include homologous recombination and site-specific recombination. In some embodiments, a specific site in the genome is chosen based on criteria such as interference with normal functioning of the cell and transcriptional activity of the site. In specific embodiments, the insertion site is chosen so that the inserted nucleic acid does not interfere with the normal functioning of the cell. In other embodiments, the insertion site is chosen so that it is, remains or becomes transcriptionaly active or inactive. In further embodiments, the transcriptional activity of the insertion site may change as the cells progress through different stages of differentiation.

In embodiments where insertion of the nucleic acid into specific regions of the genome is desired, sites with functional homology to site-specific recombination sites (pseudo sites) can be identified and used. These sites may be used to target the insertion of nucleic acids to a desired region. Sites which may be used for this purpose include, but are not limited to, those recognized by the recombinases phiC31, R4, phi80, P22, P2, 186, P4 and P1.

Genetic elements used in the practice of the invention (e.g. genetic elements for expression in cells) may be simple constructs or complex constructs. An example of a simple construct may be a single promoter and a marker gene such as a fluorescent protein. Highly complex constructs may comprise multiple promoters, reporters, selection markers, regulatory elements and/or other components. Promoters used in genetic constructs may be active only in certain cell lineages or at certain stages of development. In some embodiments, lineage specific promoters may be linked to fluorescent proteins and the expression of the fluorescent proteins used to track cells of a given lineage. In other embodiments involving cells such as stem cells, lineage specific promoters may be linked to toxic genes so that when the cell begins to differentiate down selected lineages, the toxic gene is expressed and the cell killed thereby preventing the cell from differentiating down a certain lineage or lineages.

In some embodiments, genetic elements used in the practice of the invention need not encode a protein but may encode a nucleic acid such as, for example, tRNAs, anti-sense molecules, interfering RNA and/or ribozymes etc. Interfereing RNA involves the production of double-stranded RNA, termed RNA interference (RNAi). (See, e.g., Mette et al., EMBO J., 19:5194-5201 (2000)). The double stranded region is typically from about 18 to about 30 nucleotides in length, separated by an intervening single stranded hairpin loop structure but may also be composed of two separate strands. In some embodiments the double stranded region is from 19 to 30 nucleotides, from 20 to 30 nucleotides, from 21 to 30 nucleotides, from 18 to 28 nucleotides, from 18 to 27 nucleotides, from 18 to 26 nucleotides, from 18 to 25 nucleotides in length. The double stranded region may comprise one or more (e.g., two, three or four) mismatches, as well as one or more insertion or deletion with respect to nucleotides from either of the two strands. The hairpin loop structure, when present, is typically from about 3 to about 23 nucleotides in length. In some embodiments, the hairpin loop is from 4 to 23 nucleotides, from 5 to 23 nucleotides, from 6 to 23 nucleotides, from 7 to 23 nucleotides, from 3 to 5 nucleotides, from 3 to 6 nucleotides, from 3 to 7 nucleotides, from 3 to 8 nucleotides, from 3 to 10 nucleotides, from 3 to 22 nucleotides, from 3 to 21 nucleotides, from 3 to 20 nucleotides, from 3 to 19 nucleotides, from 3 to 16 nucleotides, or from 3 to 13 nucleotides in length. Thus, the invention includes methods which involve altering the expression of genes in cells. In many instances this will be done by knocking down gene expression and can be used to alter differentiation pathways which cells follow. Vectors which may be used for knocking down gene expression include BLOCK-iT™ U6 RNAi Entry Vector (Catalog No. K4945-00) and BLOCK-iT™ Inducible H1 Lentiviral RNAi System (Catalog No. K4925-00) available from Invitrogen Corporation, Carlsbad, Calif.

Inhibitory double stranded RNA molecules may be synthesized inside of the cell or outside of the cell. Examples of double stranded RNA molecules synthesized outside of a cell include STEALTH™ RNAi molecules such as Catalog Nos. 12935-001, 12935-002 and 12935-003 available from Invitrogen Corp., Carlsbad, Calif.

Another method of silencing genes involves the production of antisense RNA/ribozymes fusions which comprise (1) antisense RNA corresponding to a target gene and (2) one or more ribozymes which cleave RNA (e.g., hammerhead ribozyme, hairpin ribozyme, delta ribozyme, Tetrahymena L-21 ribozyme, etc.).

Thus, expression products of nucleic acid molecules of the invention can be used to silence gene expression and nucleic acid molecules can be screened to identify those with activities related to gene silencing. In one non-limiting example, an RNAi molecule which knocks down expression of a gene of interest may be linked to a promoter that is linked to a certain cell type or stage of differentiation, allowing studies on the role of the RNAi targeted gene in different cell types or stages of differentiation.

In other embodiments, a detectable or selectable marker such as a fluorescent protein or antibiotic resistance gene may be linked to a differentiation state specific promoter. One use of such a system is to identify or select for cells entering a specific state of differentiation. Many different combinations of developmentally related promoters with reporter genes, selection markers and regulatory genes can be envisaged. In further embodiments, a membrane tag such as pHOOK may be operably linked to a promoter to allow selection of differentiated cells from culture using magnetic beads, FACS or other means. The invention also includes methods for using inserted genetic elements to produce cells with particular properties, methods for the regulation of gene expression by the use of RNAi molecules, methods for the regulation of cell differention, methods for selecting cells based on differentiation state, and methods for producing cells with limited differentiation potential.

Some aspects of the invention relate to methods for identifying genomic loci suitable for inserting nucleic acid molecules (e.g. heterologous nucleic acid molecules). Among other factors, a suitable genomic locus is one that is not essential for cellular function and where, in some embodiments, the genomic locus remains transcriptionaly active during cellular differentiation. Such methods can involve transfecting cells with a nucleic acid, (e.g. a nucleic acid further comprising one or more of the following: a first recombination site, a first selectable marker and a second selectable marker). In specific embodiments, cells in which a nucleic acid as described herein has been integrated into a genome may be selected by use of a first selectable marker. In further embodiments, a second nucleic acid may be constructed such that it comprises one or more of the following elements: at least one genetic element for expression in a cell, a promoter and a second recombination site. In specific embodiments, cells transfected with the a nucleic acid as described herein may be selected by use of the first selectable marker. In some embodiments, cells may be supplied with a recombinase specific for a first and/or second recombination sites such that nucleic acid is inserted into the genome of the cell. In further embodiments, cells in which a nucleic acid has been integrated into a genome may be selected by use of the second conditional selectable marker. In additional embodiments, the genomic location of one or more (e.g. two, three, four, five, seven, ten etc.) integrated nucleic acids may be mapped. In additional embodiments, cells selected with a second selectable marker, as well as other cell lines described herein, may be differentiated to each of ectoderm, endoderm and mesoderm cell types in the presence of a selection agent for the second selectable marker thereby selecting cells where the genomic site of integration remains transcriptionally active throughout differentiation. In further embodiments, a mapped genomic location of an inserted nucleic acid may be correlated with the ability to differentiate in the presence of the selection agent for the second selectable marker. This allows for identification of sites that are transcriptionally active throughout differentiation to one or more of ectoderm, endoderm or mesoderm cell types.

The insertion of nucleic acid into cells may be random or targeted. The invention is not limited by the mechanism of how a nucleic acid is inserted into a genome but possible mechanisms include homologous recombination and site-specific recombination. In some embodiments, a specific site in a genome is chosen based on criteria such as interference with normal functioning of the cell and transcriptional activity of the site. In specific embodiments, insertion sites are chosen so that inserted nucleic acids do not interfere with normal functioning of the cell. In other embodiments, insertion sites are chosen so that they remain or become transcriptionally active or inactive. In further embodiments, transcriptional activity of insertion sites may change as cells progress through different stages of differentiation.

A further aspect of the invention involves a method for directly isolating cells expressing one or more (e.g., two, three, four, five, seven, ten etc.) transfected nucleic acid molecules. The method provides transfecting a cell, such as an embryonic stem cell, with a first nucleic acid molecule. In further embodiments, the nucleic molecule may integrate into a recombination site. In specific embodiments, the recombination site may be known to possess one or more of the following properties: a pseudo recombination site, located in a genomic locus that is not essential for cellular function, and the genomic locus remains transcriptionaly active during cellular differentiation. In other embodiments, the plasmid further comprises one or more of a first recombination site which specifically recombines with the pseudo recombination site, a first selectable marker and a second conditional selectable marker. In specific embodiments, embryonic stem cells in which nucleic acid has been integrated into a genome may be selected by use of a first selectable marker and used to create a transgenic animal derived from the transfected embryonic stem cell. In further embodiments, a nucleic acid molecule comprising a promoter and a second recombination site may be constructed and transfected into cells isolated from the transgenic mouse. In specific embodiments, a recombinase specific for the first and second recombination sites is provided such that the nucleic acid is inserted into the genome of the embryonic stem cell. In some embodiments, cells which grow in the presence of the selection agent for the second selectable marker may be directly isolated. The invention includes the nucleic molecules, genetic constructs and hosts and host cells comprising the nucleic acid molecules and genetic constructs used to practice the methods of the invention. The invention also includes kits comprising one or more of nucleic molecules, genetic constructs, hosts, host cells, reagents and protocols for practicing methods of the invention.

In some embodiments, directly isolated cells may be an abundant adult stem cell type such as mesenchymal stem cells from bone marrow. Methods disclosed herein may enable stable gene transfer into early passage stem cells harvested from an animal so that there is remaining proliferative life span sufficient for further study. In other embodiments, rare cells such as neural stem cells or other tissue specific stem cells may be isolated. Methods disclosed herein allow inserting the desired genetic manipulation into many cell types in animals, in specific embodiments into every cell type in an animal. One may then isolate rare cells, such as stem cells, using reporters expressed behind tissue-specific promoters or by other means. Pools of stem cells containing desired genetic manipulations engineered at low passage may be obtained rapidly and cell quantity would be limited only be the number of animals sacrificed and the efficiency of cell selection.

The present invention also provides, in part, materials and methods for joining or combining two or more (e.g., two, three, four, five, seven, ten, twelve, fifteen, twenty, thirty, fifty, seventy-five, one hundred, two hundred, etc.) nucleic acid segments and/or nucleic acid molecules by a recombination reaction between recombination sites, at least one of which is present on each molecule and/or segment, in order to construct a nucleic acid molecule comprising all of the genetic modifications needed to insert into the cell. In embodiments of this type, one or more nucleic acid segments and/or nucleic acid molecules may comprise promoters, reporter genes, regulatory elements, genes encoding peptides or proteins, and the like. Such recombination reactions to join multiple nucleic acid segments and/or nucleic acid molecules according to the invention may be conducted in vivo (e.g., within a cell, tissue, organ or organism) or in vitro (e.g., cell-free systems). The invention also relates to hosts and host cells comprising the viral vectors and/or nucleic acid molecules of the invention. The invention also relates to kits for carrying out methods of the invention, and to compositions for carrying out methods of the invention, as well as to compositions used in and made while carrying out the methods disclosed herein.

In eukaryotic cells, DNA within chromosomes is in a highly structured environment. In order to fit within the nucleus of a cell, DNA must be tightly packed. This packing is accomplished in part by DNA molecules being associated with proteins known as histones. This DNA protein complex is referred to as chromatin. Within chromatin, DNA is wound around histone octomers in a structured manner. Chemical modifications of the histone proteins such as acetylation and methylation affect the association of the DNA molecule with the histones. The packing of DNA within chromatin strongly affects the accessibility of DNA to transcription factors and therefore strongly influences gene expression. Expressed genes are associated with regions of chromatin that are less densely packed or that have a more open structure.

The present invention further provides for compositions and methods for detecting alterations in the structure of chromatin. Chromatin structure encompasses the three dimensional arrangement of DNA and its association with proteins such as histones as well as the functional relationship between chromatin structure and gene expression. In some embodiments, genetic constructs comprising a promoter operably linked to a gene the transcription of which may be detected (e.g., a reporter gene) may be inserted into a region of the chromosome in which the chromatin structure is to be monitored. Measurement of the level of expression of the gene may serve as a marker of the structural state of the chromatin in the region of the chromosome where the genetic construct is inserted. In many embodiments, the promoter present in the genetic construct may be constitutive, in other embodiments the promoter may be developmentally regulated. A reporter gene used in the practice of the invention may be any gene that produces a product that is reacdily measured, including phenotypic markers such as β-lactamase, β-galactosidase, green fluorescent protein (GFP), yellow flourescent protein (YFP), red fluorescent protein (RFP), cyan fluorescent protein (CFP), and cell surface proteins readily detected, for example by an antibody.

In further embodiments, genetic constructs inserted into the chromosome may comprise one promoter associated with multiple detectable genes (e.g., reporter genes), multiple promoters associated with a single detectable gene or multiple promoters associated with multiple detectable genes. The use of multiple promoters may be used to ensure that the detectable gene is available throughout development even though individual promoters may only be active during certain stages of differentiation. The use of multiple detectable genes may be used to distinguish changes in chromatin structure that occur during different stages of differentiation. For example a gene for green fluorescent protein may be linked to a promoter active at an early differentiation state and a gene for a yellow fluorescent protein linked to a promoter active at a late stage of differentiation. In some embodiments multiple genetic constructs may be inserted into different regions of a chromosome or different chromosomes so that the expression of the different reporter genes reflects chromatin structure at multiple sites.

Thus, the invention provides methods and compositions for detecting alterations in chromatin structure. Such methods may involve the insertion of a gene into a chromosomal locus or monitoring expression of a gene know to reside in a particular location. As an example, hybridization assays may be used to monitor the transcription of a gene know to reside in a particular chromosomal locus.

Methods of the invention may be used to detect the alteration or structure of a chromosomal region which either allows for gene expression or inhibits gene expression. Further, few things in biology are all-or-none. Thus, the invention includes methods for detecting variations in gene expression which are based upon changes in expression levels. As an example, in some instances, high level expression (e.g., transcription) could be quantified using Northern blot analysis (e.g., slot blots) and assigned a value such as 100. Further, transcription may then be monitored under various conditions to determine whether gene expression decreases (or increases in a reverse situation). As an example, gene expression could decrease by more than half (e.g., to a value of 5, 10, 20, 30, 35, 40, 49, etc). Thus, the invention provides ratiometric methods for assessing changes in chromosomal structure. The invention further includes compositions of matter used in methods set out herein.

In some instances, the invention includes methods for screening compounds to identify those which induce or facilitate conformational changes in DNA structure. One example of such methods includes contacting a cell with particular levels of gene expression from one or more specified chromosomal loci and measurement of expression from that locus or those loci to determine whether a change in expression level occurs. In one embodiment, methods of the invention include those involving (a) detecting the level of gene expression of one or more gene in a cell located in a chromosomal locus, (b) contacting the cell with a compound to be screened for the ability to induce a structural change in the chromosomal locus, and (c) detecting the level of gene expression of the one or more gene in the cell located in the chromosomal locus. In many instances, the level of gene detected in step (a) may be compared to the level of gene expression detected in step (c). Compounds which may be screened by such methods include those which induce a change in a cell phenotype, such as compounds which stimulate, block stimulation, or inhibit G-protein coupled receptors, nuclear receptors, etc. Compounds further include hormones, cytokines, growth factors and drugs, as well as other cell signaling molecules.

Methods of the invention include the use of controls. Controls may include cells with and without the insertion of genetic constructs, constructs inserted in different locations, cells measured before and after insertion of genetic constructs, cells exposed or not exposed to a test compound(s) and cells assayed before and after exposure to a test compound(s). In some embodiments the screening assays are designed to be carried out in a high throughput manner. The cells may be assayed in multiwell plates with controls located in some wells and test cells in separate wells. Assays may have a separate control plate and test plates. Samples for analysis may be withdrawn from wells and analyzed externally, for example in a slot blot. Results of the assay may derived from a comparison of the control results to the test results.

Nucleic acid molecules prepared by methods disclosed herein may be used for any purpose known to those skilled in the art. For example, nucleic acid molecules of the invention may be used to express proteins or peptides encoded by these nucleic acid molecules and may also be used to create novel fusion proteins by expressing different nucleic acid sequences linked by the methods of the invention. Nucleic acids of the invention may also be used to produce RNA molecules that are not translated into polypeptides or proteins, for example, tRNAs, anti-sense molecules, interfering RNA and/or ribozymes.

Recombination sites for use in the methods and/or compositions of the invention may be any recognition sequence on a nucleic acid molecule that participates in a recombination reaction mediated or catalyzed by one or more recombination proteins. In those embodiments of the present invention utilizing more than one (e.g., two, three, four, five, seven, ten, twelve, fifteen, twenty, thirty, fifty, etc.) recombination sites, such recombination sites may be the same or different and may recombine with each other or may not recombine or not substantially recombine with each other. Recombination sites contemplated by the invention also include mutants, derivatives or variants of wild-type or naturally occurring recombination sites. Desired modifications can also be made to the recombination sites to include changes to the nucleotide sequence of the recombination site that cause desired sequence changes to the transcription product (e.g., mRNA, tRNA, ribozyme, etc.) and/or desired amino acid changes in the translation product (e.g., polypeptide or protein) when transcription occurs across the modified recombination site.

Exemplary recombination sites used in accordance with the invention include att sites, frt sites, dif sites, psi sites, cer sites, and lox sites or mutants, derivatives and variants thereof (or combinations thereof). Recombination sites contemplated by the invention also include portions of such recombination sites. Depending on the recombination site specificity used, the invention allows directional linking of nucleic acid molecules to provide desired orientations of the linked molecules or non-directional linking to produce random orientations of the linked molecules.

In certain embodiments, recombination proteins used in the practice of the invention comprise one or more proteins selected from the group consisting of Cre, Int, IHF, Xis, Flp, Fis, Hin, Gin, CM, Tn3 resolvase, TndX, XerC, XerD, and phiC31. In specific embodiments, the recombination sites comprise one or more recombination sites selected from the group consisting of lox sites; psi sites; dif sites; cer sites; frt sites; att sites; and mutants, variants, and derivatives of these recombination sites that retain the ability to undergo recombination.

Other embodiments may be a method for identifying genes that effect cell performance, the method comprising: a) transfecting the population of cells with a first nucleic acid molecule, said nucleic acid molecule further comprising a first recombination site, a first selectable marker and a second selectable marker; b) selecting cells from the population in which the first nucleic acid has been integrated into the genome; c) transfecting the cells selected by use of the first selectable marker with a second nucleic acid comprising at least one genetic element which corrects the genetic defect, a promoter and a second recombination site and providing to the selected cells a recombinase specific for the first and second recombination sites such that the second nucleic acid is inserted into the genome of the cell by site-specific recombination; d) selecting cells in which the second nucleic acid has been integrated into the genome; and e) determining bioproduction of selected cells.

Compositions, methods and kits of the invention may be prepared and carried out using a phage-lambda site-specific recombination system, such as with the GATEWAY™ Recombinational Cloning System available from Invitrogen Corporation, Carlsbad, Calif. The GATEWAY™ Technology Instruction Manual (catalog numbers 12535-019 and 12535-027 Version E, Invitrogen Corporation, Carlsbad, Calif.) describes in more detail this system and is incorporated herein by reference in its entirety.

### BRIEF DESCRIPTION OF THE DRAWINGS

FIG. 1 shows a schematic representation of embodiments of the invention where embryonic stem cells are used.

FIG. 2 shows a schematic representation of embodiments of the invention where adult stem cells are used.

FIG. 3 shows a schematic representation of how differentiation or tissue specific promoters may be used to control the differentiation of genetically modified cells.

FIG. 4a shows a schematic representation of a TA cloning reaction.

FIG. 4b shows a schematic representation of primer selection to add modified attB sites to entry clones.

FIG. 4c shows a schematic representation of the BP recombination reaction for assembling an entry clone.

FIG. 4d shows a schematic representation of the LR recombination reaction for assembling multiple entry clones into one entry vector.

FIG. 5 shows six examples of modified attB sites. The underlined portions of the sequence indicate the core sequence that determines specificity.

FIG. 6 illustrates the use of intermediate destination vectors for the construction of high order assemblies.

FIG. 7 illustrates one non-limiting example of how successful insertion of an expression vector can be selected for by activation of a previously inactive antibiotic resistance gene.

FIG. 8 Plasmid map of hOKG Real plasmid used for transformation of human embryonic stem cells.

FIG. 9 shows the cellular expression pattern of the Oct-4 and GFP proteins in transfected BGO1v cells.

FIG. 10 shows the fluorescence profile of the Oct-4/GFP transfected BGO1v cells.

FIG. 11 shows the fluorescence profile of the Oct-4/GFP transfected BGO1v cells at day 0 and at 21 days after differentiation was initiated.

FIG. 12 Illustrates the strategy and plasmids used in the study. Multisite gateway technology was used to assemble the phOG construct from the appropriate Entry vectors and the Destination vector pB2H1-DEST. This plasmid was then used to transfect variant human embryonic stem cell lines (hESC) along with a plasmid expressing the phiC31 integrase (pCMV-phiC31 Int). Co-transfection results in integration of the expression plasmid into pseudo attP sites in the genome.

FIG. 13 PhiC31 integrase-mediated pseudo sites obtained in hESC were analyzed along with the native phiC31 attP for the presence of a common motif by using the MEME motif finder to analyze 100 bp of genomic DNA surrounding the observed crossover site. A. Presence of the principal motif in the pseudo sites. The 26 bp attP motif appeared in all 24 of the included sequences close to the area of the observed crossover (indicated by the 50 bp midpoint of the sequence). The consensus sequence is symmetrical about the core and contains inverted repeats (arrows) extending over the length of the consensus. B. A sequence logo diagram for the MEME motif. The probability of a given base occurring at a position is represented by the size of the letter.

FIG. 14 Clones resulting from transfection of GFP expression plasmid and phiC31 integrase were picked, expanded and their integration sites were mapped. Representative hOct4-GFP clones derived from BG01v and SA002 cells and an EF1α-GFP clone derived from BG01v were analyzed for expression of Oct4 (by immunostaining, red) and GFP (fluorescence, green). The cells are counter-stained with DAPI (blue). B. Panel I shows the expression of GFP driven by either the Oct4 promoter or the EF1α promoter. EF1 α-driven expression is typically an order of magnitude higher than Oct4-driven expression. Panels II and III show long-term expression of GFP in transgenic lines. PhiC31 integrase-derived cells were cultured in the presence of the selectable marker for an extended period, and GFP expression was analyzed by FACS at regular intervals. Typically, the cells were cultured for at least 10 passages, which is approximately 4 to 5 weeks.

FIG. 15 Three BG01v-derived Oct4-GFP lines (YA06, YA15 and YA18) and one SA002-derived Oct4-GFP line (YB1403) were allowed to form embryoid bodies to characterize the differentiation potential of phiC31 integrase-derived lines. Differentiation into the endodermal (α-Fetoprotein), mesodermal (Muscle-specific actin and Brachyury) and ectodermal (βIII-Tubulin and Nestin) lineages was analyzed by immunostaining with specific antibodies (Red). The cells are counter-stained with DAPI (blue).

FIG. 16 The BG01v-derived Oct4-GFP clones, YA06, YA15 and YA18 and the BG01v-derived EF1α-GFP clone EG101 were allowed to form embryoid bodies for 21 days under selection and GFP expression was analyzed by FACS. The red curves indicate a control line that did not express GFP, green curves indicate undifferentiated cells, and blue curves indicate EBs derived from those cells. GFP expression is shut down in all three Oct4-GFP clones upon formation of embryoid bodies, as opposed to the EF1α-GFP clone.

FIG. 17 shows a schematic representation of embodiments for generation of a retarget line platform.

FIG. 18 shows a schematic representation for screening of cell performance enhancing genes.

FIG. 19 shows a schematic representation for screen of cells for bioproductions and drug discovery.

FIG. 20 shows the effect of a TRPM8 retargeted pool in Hek293 on calcium expression.

FIG. 21 shows a comparison of calcium expression in a CCKAR retargeted pool vs. a bla cone in HEK 293.

FIG. 22 shows results of a CHOS R4 line retargeted with a GFP gene.

### DETAILED DESCRIPTION OF THE INVENTION

In the description that follows, a number of terms used in recombinant nucleic acid technology are utilized extensively. In order to provide a clear and more consistent understanding of the specification and claims, including the scope to be given such terms, the following definitions are provided.

Stem Cell: As used herein, the term “stem cell” refers to an unspecialized cell capable of developing into a variety of specialized cells and tissues. Stem cells can be broadly divided into embryonic stem cells and adult stem cells. Embryonic stem cells are found in very early embryos and are derived from a group of cells called the inner cell mass, a part of blastocyst. Embryonic stem cells are self-renewing and can form all cell types found in the body (pluripotent). Adult stem cells may be obtained from, among other sources, blood, bone marrow, brain, pancreas, and fat of adult bodies. Adult stem cells may renew themselves and differentiate to give rise to all the specialized cell types of the tissue from which it originated and potentially cell types associated with other tissues (multipotent). In some embodiments the stem cells may be of plant origin. Stem cells are known to occur in a number of locations in the seed and developing or adult plant. Plant stem cells may be from any of the tissues in which stem cells are present. Examples include stem cells from the apical or root meristems. In some embodiments, the stem cells are from an agriculturally important plant. The plant may be, for example, maize, wheat, rice, potato, an edible fruit-bearing plant or other commercially farmed plant.

Gene: As used herein, the term “gene” refers to a nucleic acid that contains information necessary for expression of a polypeptide, protein, or untranslated RNA (e.g., rRNA, tRNA, anti-sense RNA). When the gene encodes a protein, it includes the promoter and the open reading frame sequence (ORF), as well as other sequences involved in expression of the protein. When the gene encodes an untranslated RNA, it includes the promoter and the nucleic acid that encodes the untranslated RNA.

Host: As used herein, the term “host” refers to any prokaryotic or eukaryotic (e.g., mammalian, insect, yeast, plant, avian, animal, etc.) organism that is a recipient of a replicable expression vector, cloning vector or any nucleic acid molecule. The nucleic acid molecule may contain, but is not limited to, a sequence of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Sambrook, et al., Molecular Cloning: A Laboratory Manual, Cold Spring Harbor Laboratory, Cold Spring Harbor, N.Y.

Promoter: As used herein, a promoter is an example of a transcriptional regulatory sequence, and is specifically a nucleic acid generally described as the 5′-region of a gene located proximal to the start codon or nucleic acid that encodes untranslated RNA. The transcription of an adjacent nucleic acid segment is initiated at or near the promoter. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

Activity of a given promoter may be limited to a specific stage of development, a certain lineage or type of cell or to a particular differentiation state. Such promoters may collectively be referred to as developmental promoters.

Target Nucleic Acid Molecule: As used herein, the phrase “target nucleic acid molecule” refers to a nucleic acid segment of interest, preferably nucleic acid that is to be acted upon using the compounds and methods of the present invention. Such target nucleic acid molecules may contain one or more (e.g., two, three, four, five, seven, ten, twelve, fifteen, twenty, thirty, fifty, etc.) genes or one or more portions of genes.

Recombinases: As used herein, the term “recombinases” is used to refer to the protein that catalyzes strand cleavage and re-ligation in a recombination reaction. Site-specific recombinases are proteins that are present in many organisms (e.g., viruses and bacteria) and have been characterized as having both endonuclease and ligase properties. These recombinases (along with associated proteins in some cases) recognize specific sequences of bases in a nucleic acid molecule and exchange the nucleic acid segments flanking those sequences. The recombinases and associated proteins are collectively referred to as “recombination proteins” (see, e.g., Landy, A., Current Opinion in Biotechnology 3:699-707 (1993)). Examples of recombination proteins include but are not limited to Cre, Int, IHF, Xis, Flp, F is, Hin, Gin, phiC31, R4, BxB1, CM, Tn3 resolvase, TndX, XerC, XerD, TnpX, Hjc, SpCCE1, and ParA.

Numerous recombination systems from various organisms have been described. See, e.g., Hoess, et al., Nucleic Acids Research 14(6):2287 (1986); Abremski, et al., J. Biol. Chem. 261(1):391 (1986); Campbell, J. Bacteriol. 174(23):7495 (1992); Qian, et al., J. Biol. Chem. 267(11):7794 (1992); Araki, et al., J. Mol. Biol. 225(1):25 (1992); Maeser and Kahnmann, Mol. Gen. Genet. 230:170-176) (1991); Esposito, et al., Nucl. Acids Res. 25(18):3605 (1997). Many of these belong to the integrase family of recombinases (Argos, et al., EMBO J. 5:433-440 (1986); Voziyanov, et al., Nucl. Acids Res. 27:930 (1999)). Perhaps the best studied of these are the Integrase/att system from bacteriophage λ (Landy, A. Current Opinions in Genetics and Devel. 3:699-707 (1993)), the Cre/loxP system from bacteriophage P1 (Hoess and Abremski (1990) In Nucleic Acids and Molecular Biology, vol. 4. Eds.: Eckstein and Lilley, Berlin-Heidelberg: Springer-Verlag; pp. 90-109), and the FLP/FRT system from the Saccharomyces cerevisiae 2μ circle plasmid (Broach, et al., Cell 29:227-234 (1982)).

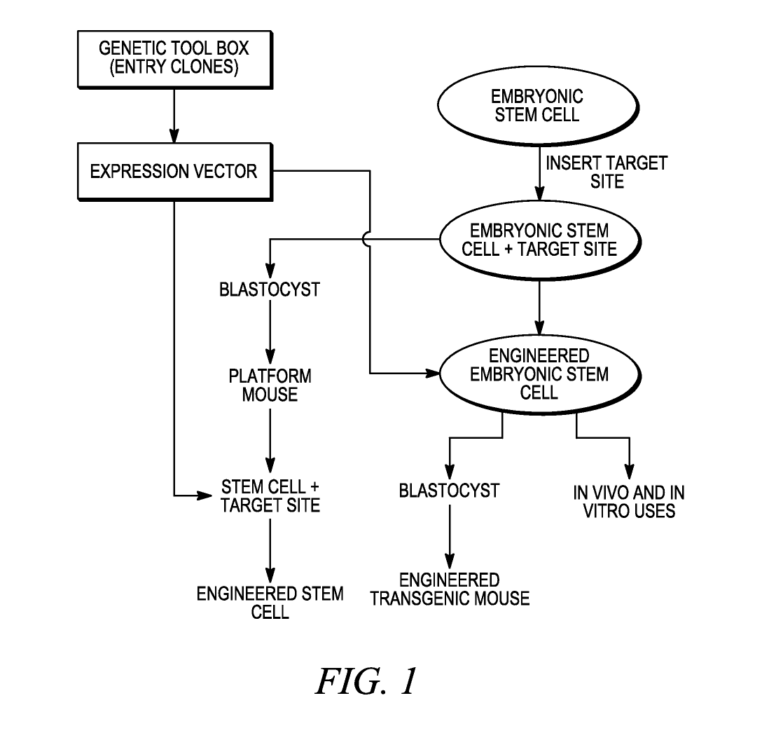

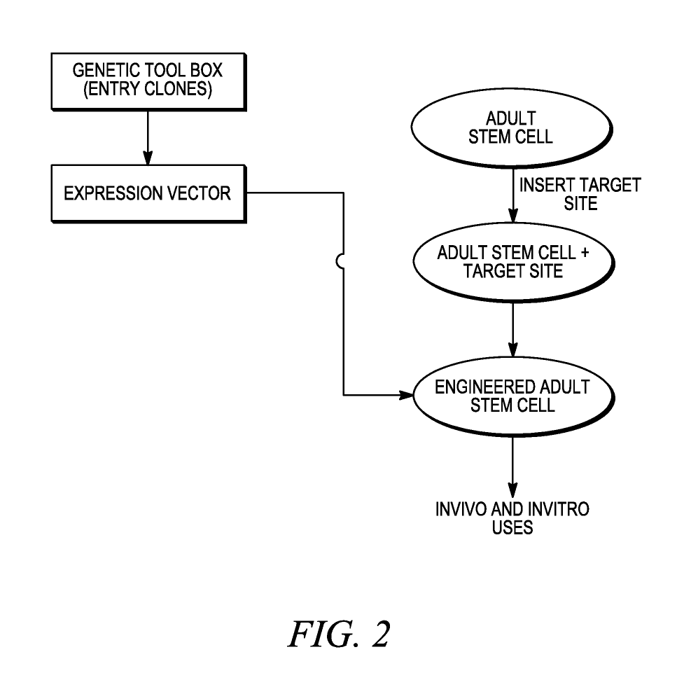

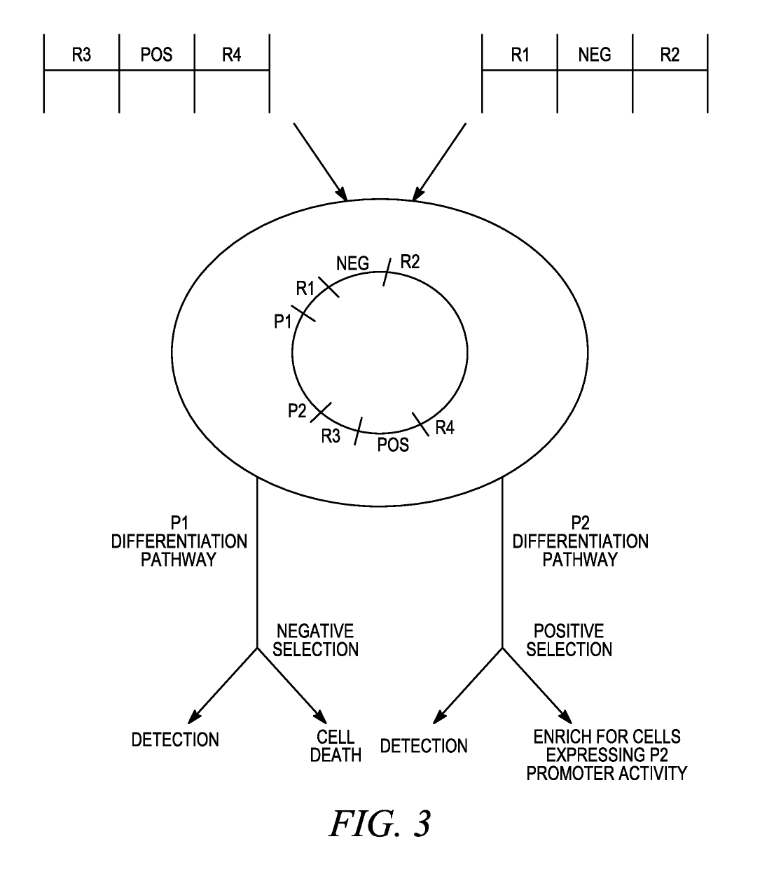

Recombination Site: A used herein, the phrase “recombination site” refers to a recognition sequence on a nucleic acid molecule that participates in an integration/recombination reaction by recombination proteins. Recombination sites are discrete sections or segments of nucleic acid on the participating nucleic acid molecules that are recognized and bound by a site-specific recombination protein during the initial stages of integration or recombination. For example, the recombination site for Cre recombinase is loxP, which is a 34 base pair sequence comprised of two 13 base pair inverted repeats (serving as the recombinase binding sites) flanking an 8 base pair core sequence (see FIG. 1 of Sauer, B., Curr. Opin. Biotech. 5:521-527 (1994)). Other examples of recombination sites include the attB, attP, attL, and attR sequences described in U.S. provisional patent applications 60/136,744, filed May 28, 1999, and 60/188,000, filed Mar. 9, 2000, and in co-pending U.S. patent application Ser. Nos. 09/517,466 and 09/732,91, all of which are specifically incorporated herein by reference, and mutants, fragments, variants and derivatives thereof, which are recognized by the recombination protein λ Int and by the auxiliary proteins integration host factor (IHF), FIS and excisionase (Xis) (see Landy, Curr. Opin. Biotech. 3:699-707 (1993)).

Recombination sites may be added to molecules by any number of known methods. For example, recombination sites can be added to nucleic acid molecules by blunt end ligation, PCR performed with fully or partially random primers, or inserting the nucleic acid molecules into an vector using a restriction site flanked by recombination sites.

Recombinational Cloning: As used herein, the phrase “recombinational cloning” refers to a method, such as that described in U.S. Pat. Nos. 5,888,732; 6,143,557; 6,171,861; 6,270,969; and 6,277,608 (the contents of which are fully incorporated herein by reference), whereby segments of nucleic acid molecules or populations of such molecules are exchanged, inserted, replaced, substituted or modified, in vitro or in vivo. Preferably, such cloning method is an in vitro method.

Cloning systems that utilize recombination at defined recombination sites have been previously described in U.S. Pat. No. 5,888,732, U.S. Pat. No. 6,143,557, U.S. Pat. No. 6,171,861, U.S. Pat. No. 6,270,969, and U.S. Pat. No. 6,277,608, and in pending U.S. application Ser. No. 09/517,466 filed Mar. 2, 2000, and in published United States application nos. 2002/0007051-A1 and 2004/0229229, all assigned to Invitrogen Corporation, Carlsbad, Calif., the disclosures of which are specifically incorporated herein in their entirety. In brief, the Gateway™ Cloning System described in these patents and applications utilizes vectors that contain at least one recombination site to clone desired nucleic acid molecules (sometimes referred to as entry clones) in vivo or in vitro. In some embodiments, the system utilizes vectors that contain at least two different site-specific recombination sites that may be based on the bacteriophage lambda system (e.g., att1 and att2) that are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the Gateway™ system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

Mutating specific residues in the core region of the att site can generate a large number of different att sites. As with the att1 and att2 sites utilized in Gateway™, each additional mutation potentially creates a novel att site with unique specificity that will recombine only with its cognate partner att site bearing the same mutation and will not cross-react with any other mutant or wild-type att site. Novel mutated att sites (e.g., attB 1-10, attP 1-10, attR 1-10 and attL 1-10) are described in previous patent application Ser. No. 09/517,466, filed Mar. 2, 2000, which is specifically incorporated herein by reference. Other recombination sites having unique specificity (i.e., a first site will recombine with its corresponding site and will not recombine or not substantially recombine with a second site having a different specificity) may be used to practice the present invention. Examples of suitable recombination sites include, but are not limited to, loxP sites; loxP site mutants, variants or derivatives such as loxP511 (see U.S. Pat. No. 5,851,808); frt sites; frt site mutants, variants or derivatives; dif sites; dif site mutants, variants or derivatives; psi sites; psi site mutants, variants or derivatives; cer sites; and cer site mutants, variants or derivatives.

Repression Cassette: As used herein, the phrase “repression cassette” refers to a nucleic acid segment that contains a repressor or a selectable marker present in the subcloning vector.

Selectable Marker: As used herein, the phrase “selectable marker” refers to a nucleic acid segment that allows one to select for or against a molecule (e.g., a replicon) or a cell that contains it and/or permits identification of a cell or organism that contains or does not contain the nucleic acid segment. Frequently, selection and/or identification occur under particular conditions and do not occur under other conditions.

Markers can encode an activity, such as, but not limited to, production of RNA, peptide, or protein, or can provide a binding site for RNA, peptides, proteins, inorganic and organic compounds or compositions and the like. Examples of selectable markers include but are not limited to: (1) nucleic acid segments that encode products that provide resistance against otherwise toxic compounds (e.g., antibiotics); (2) nucleic acid segments that encode products that are otherwise lacking in the recipient cell (e.g., tRNA genes, auxotrophic markers); (3) nucleic acid segments that encode products that suppress the activity of a gene product; (4) nucleic acid segments that encode products that can be readily identified (e.g., phenotypic markers such as β-lactamase, β-galactosidase, green fluorescent protein (GFP), yellow flourescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; and/or (11) nucleic acid segments that encode products that either are toxic (e.g., Diphtheria toxin) or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, etc.).

Selection and/or identification may be accomplished using techniques well known in the art. For example, a selectable marker may confer resistance to an otherwise toxic compound and selection may be accomplished by contacting a population of host cells with the toxic compound under conditions in which only those host cells containing the selectable marker are viable. In another example, a selectable marker may confer sensitivity to an otherwise benign compound and selection may be accomplished by contacting a population of host cells with the benign compound under conditions in which only those host cells that do not contain the selectable marker are viable. A selectable marker may make it possible to identify host cells containing or not containing the marker by selection of appropriate conditions. In one aspect, a selectable marker may enable visual screening of host cells to determine the presence or absence of the marker. For example, a selectable marker may alter the color and/or fluorescence characteristics of a cell containing it. This alteration may occur in the presence of one or more compounds, for example, as a result of an interaction between a polypeptide encoded by the selectable marker and the compound (e.g., an enzymatic reaction using the compound as a substrate). Such alterations in visual characteristics can be used to physically separate the cells containing the selectable marker from those not contain it by, for example, fluorescent activated cell sorting (FACS).

Multiple selectable markers may be simultaneously used to distinguish various populations of cells. For example, a nucleic acid molecule of the invention may have multiple selectable markers, one or more of which may be removed from the nucleic acid molecule by a suitable reaction (e.g., a recombination reaction). After the reaction, the nucleic acid molecules may be introduced into a host cell population and those host cells comprising nucleic acid molecules having all of the selectable markers may be distinguished from host cells comprising nucleic acid molecules in which one or more selectable markers have been removed (e.g., by the recombination reaction). For example, a nucleic acid molecule of the invention may have a blasticidin resistance marker outside a pair of recombination sites and a β-lactamase encoding selectable marker inside the recombination sites. After a recombination reaction and introduction of the reaction mixture into a cell population, cells comprising any nucleic acid molecule can be selected for by contacting the cell population with blasticidin. Those cell comprising a nucleic acid molecule that has undergone a recombination reaction can be distinguished from those containing an unreacted nucleic acid molecules by contacting the cell population with a fluorogenic β-lactamase substrate as described below and observing the fluorescence of the cell population. Optionally, the desired cells can be physically separated from undesirable cells, for example, by FACS.

In a specific embodiment of the invention, a selectable marker may be a nucleic acid sequence encoding a polypeptide having an enzymatic activity (e.g., β-lactamase activity). Assays for β-lactamase activity are known in the art. U.S. Pat. Nos. 5,955,604, issued to Tsien, et al. Sep. 21, 1999, 5,741,657 issued to Tsien, et al., Apr. 21, 1998, 6,031,094, issued to Tsien, et al., Feb. 29, 2000, 6,291,162, issued to Tsien, et al., Sep. 18, 2001, and 6,472,205, issued to Tsien, et al. Oct. 29, 2002, disclose the use of β-lactamase as a reporter gene and fluorogenic substrates for use in detecting β-lactamase activity and are specifically incorporated herein by reference. In addition photon reducing agents may be used in conjunction with the fluorogenic substrates. Suitable photon reducing agents include those described in U.S. Pat. No. 7,067,324 which is specifically incorporated herein by reference. Commercially available photon reducing agents are described in the CELLSENSOR™ Assay Protocol Manual (Catalog No. K1097) incorporated herein by reference in its entirety, available from Invitrogen Corp., Carlsbad, Calif. In one embodiment of the invention, a selectable marker may be a nucleic acid sequence encoding a polypeptide having β-lactamase activity and desired host cells may be identified by assaying the host cells for β-lactamase activity.

A β-lactamase catalyzes the hydrolysis of a β-lactam ring. Those skilled in the art will appreciate that the sequences of a number of polypeptides having β-lactamase activity are known. In addition to the specific β-lactamases disclosed in the Tsien, et al. patents listed above, any polypeptide having β-lactamase activity is suitable for use in the present invention.

β-lactamases are classified based on amino acid and nucleotide sequence (Ambler, R. P., Phil. Trans. R. Soc. Lond. [Ser.B.] 289: 321-331 (1980)) into classes A-D. Class A β-lactamases possess a serine in the active site and have an approximate weight of 29 kD. This class contains the plasmid-mediated TEM β-lactamases such as the RTEM enzyme of pBR322. Class B β-lactamases have an active-site zinc bound to a cysteine residue. Class C enzymes have an active site serine and a molecular weight of approximately 39 kD, but have no amino acid homology to the class A enzymes. Class D enzymes also contain an active site serine. Representative examples of each class are provided below with the accession number at which the sequence of the enzyme may be obtained in the indicated database.

Site-Specific Recombinase: As used herein, the phrase “site-specific recombinase” refers to a type of recombinase that typically has at least the following four activities (or combinations thereof): (1) recognition of specific nucleic acid sequences; (2) cleavage of said sequence or sequences; (3) topoisomerase activity involved in strand exchange; and (4) ligase activity to reseal the cleaved strands of nucleic acid (see Sauer, B., Current Opinions in Biotechnology 5:521-527 (1994)). Conservative site-specific recombination is distinguished from homologous recombination and transposition by a high degree of sequence specificity for both partners. The strand exchange mechanism involves the cleavage and rejoining of specific nucleic acid sequences in the absence of DNA synthesis (Landy, A. (1989) Ann. Rev. Biochem. 58:913-949).

In some embodiments of the invention, a selectable marker may be a nucleic acid sequence encoding a polypeptide which is an integral membrane protein that may act as a cellular tag. (Further examples of these embodiments may be found in U.S. Pat. No. 6,017,754 incorporated herein by reference.) In these embodiments, the polypeptide may encode a single chain antibody fused with a PDGF transmembrane domain and a secretion leader sequence. This polypeptide may be expressed under the control of various promoter types as mentioned above, the protein may be inserted into the cell membrane and may display the single chain antibody on the extracellular surface. Tagged cells may then be selected from the total population by incubation with magnetic beads coated with the specific antigen for the single chain antibody (phOx).

Suppressor tRNAs: A tRNA molecule that results in the incorporation of an amino acid in a polypeptide in a position corresponding to a stop codon in the mRNA being translated.

Homologous Recombination: As used herein, the phrase “homologous recombination” refers to the process in which nucleic acid molecules with similar nucleotide sequences associate and exchange nucleotide strands. A nucleotide sequence of a first nucleic acid molecule that is effective for engaging in homologous recombination at a predefined position of a second nucleic acid molecule will therefore have a nucleotide sequence that facilitates the exchange of nucleotide strands between the first nucleic acid molecule and a defined position of the second nucleic acid molecule. Thus, the first nucleic acid will generally have a nucleotide sequence that is sufficiently complementary to a portion of the second nucleic acid molecule to promote nucleotide base pairing.

Homologous recombination requires homologous sequences in the two recombining partner nucleic acids but does not require any specific sequences. As indicated above, site-specific recombination that occurs, for example, at recombination sites such as att sites, is not considered to be “homologous recombination,” as the phrase is used herein.

Vector: As used herein, the term “vector” refers to a nucleic acid molecule (preferably DNA) that provides a useful biological or biochemical property to an insert. A vector may be a nucleic acid molecule comprising all or a portion of a viral genome. Examples include plasmids, phages, autonomously replicating sequences (ARS), centromeres, and other sequences that are able to replicate or be replicated in vitro or in a host cell, or to convey a desired nucleic acid segment to a desired location within a host cell. A vector can have one or more recognition sites (e.g., two, three, four, five, seven, ten, etc. recombination sites, restriction sites, and/or topoisomerases sites) at which the sequences can be manipulated in a determinable fashion without loss of an essential biological function of the vector, and into which a nucleic acid fragment can be spliced in order to bring about its replication and cloning. Vectors can further provide primer sites (e.g., for PCR), transcriptional and/or translational initiation and/or regulation sites, recombinational signals, replicons, selectable markers, etc. Clearly, methods of inserting a desired nucleic acid fragment that do not require the use of recombination, transpositions or restriction enzymes (such as, but not limited to, uracil N-glycosylase (UDG) cloning of PCR fragments (U.S. Pat. Nos. 5,334,575 and 5,888,795, both of which are entirely incorporated herein by reference), TA cloning, and the like) can also be applied to clone a fragment into a cloning vector to be used according to the present invention. The cloning vector can further contain one or more selectable markers (e.g., two, three, four, five, seven, ten, etc.) suitable for use in the identification of cells transformed with the cloning vector.

Subcloning Vector: As used herein, the phrase “subcloning vector” refers to a cloning vector comprising a circular or linear nucleic acid molecule that includes, preferably, an appropriate replicon. In the present invention, the subcloning vector can also contain functional and/or regulatory elements that are desired to be incorporated into the final product to act upon or with the cloned nucleic acid insert. The subcloning vector can also contain a selectable marker (preferably DNA).

Primer: As used herein, the term “primer” refers to a single stranded or double stranded oligonucleotide that is extended by covalent bonding of nucleotide monomers during amplification or polymerization of a nucleic acid molecule (e.g., a DNA molecule). In one aspect, the primer may be a sequencing primer (for example, a universal sequencing primer). In another aspect, the primer may comprise a recombination site or portion thereof.

Template: As used herein, the term “template” refers to a double stranded or single stranded nucleic acid molecule that is to be amplified, synthesized or sequenced. In the case of a double-stranded DNA molecule, denaturation of its strands to form a first and a second strand is preferably performed before these molecules may be amplified, synthesized or sequenced, or the double stranded molecule may be used directly as a template. For single stranded templates, a primer complementary to at least a portion of the template hybridizes under appropriate conditions and one or more polypeptides having polymerase activity (e.g., two, three, four, five, or seven DNA polymerases and/or reverse transcriptases) may then synthesize a molecule complementary to all or a portion of the template. Alternatively, for double stranded templates, one or more transcriptional regulatory sequences (e.g., two, three, four, five, seven or more promoters) may be used in combination with one or more polymerases to make nucleic acid molecules complementary to all or a portion of the template. The newly synthesized molecule, according to the invention, may be of equal or shorter length compared to the original template. Mismatch incorporation or strand slippage during the synthesis or extension of the newly synthesized molecule may result in one or a number of mismatched base pairs. Thus, the synthesized molecule need not be exactly complementary to the template. Additionally, a population of nucleic acid templates may be used during synthesis or amplification to produce a population of nucleic acid molecules typically representative of the original template population.

Incorporating: As used herein, the term “incorporating” means becoming a part of a nucleic acid (e.g., DNA) molecule or primer.

Library: As used herein, the term “library” refers to a collection of nucleic acid molecules (circular or linear). In one embodiment, a library may comprise a plurality of nucleic acid molecules (e.g., two, three, four, five, seven, ten, twelve, fifteen, twenty, thirty, fifty, one hundred, two hundred, five hundred one thousand, five thousand, or more), that may or may not be from a common source organism, organ, tissue, or cell. In another embodiment, a library is representative of all or a portion or a significant portion of the nucleic acid content of an organism (a “genomic” library), or a set of nucleic acid molecules representative of all or a portion or a significant portion of the expressed nucleic acid molecules (a cDNA library or segments derived therefrom) in a cell, tissue, organ or organism. A library may also comprise nucleic acid molecules having random sequences made by de novo synthesis, mutagenesis of one or more nucleic acid molecules, and the like. Such libraries may or may not be contained in one or more vectors (e.g., two, three, four, five, seven, ten, twelve, fifteen, twenty, thirty, fifty, etc.).

Amplification: As used herein, the term “amplification” refers to any in vitro method for increasing the number of copies of a nucleic acid molecule with the use of one or more polypeptides having polymerase activity (e.g., one, two, three, four or more nucleic acid polymerases or reverse transcriptases). Nucleic acid amplification results in the incorporation of nucleotides into a DNA and/or RNA molecule or primer thereby forming a new nucleic acid molecule complementary to a template. The formed nucleic acid molecule and its template can be used as templates to synthesize additional nucleic acid molecules. As used herein, one amplification reaction may consist of many rounds of nucleic acid replication. DNA amplification reactions include, for example, polymerase chain reaction (PCR). One PCR reaction may consist of 5 to 100 cycles of denaturation and synthesis of a DNA molecule.

Nucleotide: As used herein, the term “nucleotide” refers to a base-sugar-phosphate combination. Nucleotides are monomeric units of a nucleic acid molecule (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [α-S]dATP, 7-deaza-dGTP and 7-deaza-dATP. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

Nucleic Acid Molecule: As used herein, the phrase “nucleic acid molecule” refers to a sequence of contiguous nucleotides (riboNTPs, dNTPs, ddNTPs, or combinations thereof) of any length. A nucleic acid molecule may encode a full-length polypeptide or a fragment of any length thereof, or may be non-coding. As used herein, the terms “nucleic acid molecule” and “polynucleotide” may be used interchangeably and include both RNA and DNA.

Oligonucleotide: As used herein, the term “oligonucleotide” refers to a synthetic or natural molecule comprising a covalently linked sequence of nucleotides that are joined by a phosphodiester bond between the 3′ position of the pentose of one nucleotide and the 5′ position of the pentose of the adjacent nucleotide.

Polypeptide: As used herein, the term “polypeptide” refers to a sequence of contiguous amino acids of any length. The terms “peptide,” “oligopeptide,” or “protein” may be used interchangeably herein with the term “polypeptide.”

Hybridization: As used herein, the terms “hybridization” and “hybridizing” refer to base pairing of two complementary single-stranded nucleic acid molecules (RNA and/or DNA) to give a double stranded molecule. As used herein, two nucleic acid molecules may hybridize, although the base pairing is not completely complementary. Accordingly, mismatched bases do not prevent hybridization of two nucleic acid molecules provided that appropriate conditions, well known in the art, are used. In some aspects, hybridization is said to be under “stringent conditions.” By “stringent conditions,” as the phrase is used herein, is meant overnight incubation at 42° C. in a solution comprising: 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C.

Other terms used in the fields of recombinant nucleic acid technology and molecular and cell biology as used herein will be generally understood by one of ordinary skill in the applicable arts.

The invention may be used to genetically modify cells, for example stem cells or progenitor cells. The invention may also be used to induce in vivo stem cell or progenitor cell mobilization, migration, integration, proliferation and differentiation. Stem cells may be pluripotent, that is they may be capable of giving rise to a plurality of different differentiated cell types. In some cases stem cells may be totipotent, that is they may be capable of giving rise to all of the different cell types of the organism that they are derived from. The invention is applicable to totipotent, pluripotent or multipotent stem cells. A progenitor cell is an early descendant of a stem cell that can differentiate, but cannot renew itself. Progenitor cells are more differentiated than stem cells.

In some embodiments, the invention is used to genetically modify adult stem cells. Adult stem cells are known to occur in a number of locations in the animal body. Stem cells genetically modified or obtained by the present invention may be those from any of organs and tissues in which stem cells are present. Examples include stem cells from bone marrow, haematopoietic system, neuronal system, brain, muscle stem cells or umbilical cord stem cells. Stem cells may in particular be bone marrow stromal stem cells, neuronal stem cells or haematopoietic stem cells, in some embodiments they may be bone marrow stromal stem cells or neuronal stem cells. In particular when the methods disclosed herein are used to genetically modify a stem cell, the stem cell may be a bone marrow stromal cell.

Stem cells used in the practice of the invention may be plant or animal stem cells.

In some embodiments, stem cells will be animal stem cells and preferably mammalian stem cells. In some embodiments, stem cells may be human stem cells. Alternatively, stem cells may be from a non-human animal and in particular from a non-human mammal. Stem cells may be those of a domestic animal or an agriculturally important animal. An animal may, for example, be a sheep, pig, cow, horse, bull, or poultry bird or other commercially-farmed animal. An animal may be a dog, cat, or bird and in particular from a domesticated animal. An animal may be a non-human primate such as a monkey. For example, a primate may be a chimpanzee, gorilla, or orangutan. Stem cells may be rodent stem cells. For example, stem cells may be from a mouse, rat, or hamster.

In another embodiment, stem cells will be plant stem cells. Stem cells are known to occur in a number of locations in the seed and developing or adult plant. Stem cells genetically modified or obtained in the present invention may be those from any of the tissues in which stem cells are present. Examples include stem cells from the apical or root meristems. In one embodiment, the stem cells are from an agriculturally important plant. Plants may, for example, be maize, wheat, rice, potato, an edible fruit-bearing plant or other commercially farmed plant.

In many cases genetically modified stem cells may be intended to treat a subject, or in the manufacture of medicaments. In such cases stem cells may be from the intended recipient. In other cases stem cells may originate from a different subject, but be chosen to be immunologically compatible with the intended recipient. In some cases stem cells may be from a relation of the intended recipient such as a sibling, half-sibling, cousin, parent or child, and in particular from a sibling. Stem cells may be from an unrelated subject who has been tissue typed and found to have a immunological profile which will result in no immune response or only a low immune response from the intended recipient which is not detrimental to the subject. However, in many cases the stem cells, may be from an unrelated subject as the invention may be used to render the stem cell immunologically compatible with the intended recipient. For example, stem cell and the recipient may or may not have a histocompatible haplotypes (e.g. HLA haplotypes).

In some cases stem cells may be embryonic stem cells, fetal stem cells, neonatal stem cells, or juvenile stem cells. Embryonic, fetal, neonatal, or juvenile stem cells may be multipotent stems cells and particularly pluripotent stem cells. Cells may be from any stage or sub-stage of development, in particular they may be derived from the inner cell mass of a blastocyst (e.g. embryonic stem cells). Embryonic, fetal, neonatal or juvenile stem cells may be from, or derived from, any of the organisms mentioned herein. Embryonic, fetal, neonatal or juvenile stem cells may be human stem cells or non-human stem cells and in particular non-human animal stem cells (e.g. a non-human primate). Embryonic, fetal, neonatal or juvenile stem cells may be rodent stem cells and may in particular be mouse embryonic stem cells. In some cases the embryonic, fetal, neonatal or juvenile stem cells may be recovered and then used in the manufacture of medicaments to treat the same subject, typically at some stage in their life. In one embodiment, where embryonic, fetal, neonatal or juvenile stem cells are employed, they will be from already established fetal, embryonic, neonatal or juvenile stem cell lines. This will particularly be the case for human cells. In some cases stem cells may be obtained from, or derived from, extra-embryonic tissues. Stem cells may be obtained from umbilical cord and in particular from umbilical cord blood.

The invention is also applicable to stem cell lines. Stem cell lines are generally stem cell populations that have been isolated from an organism and maintained in culture. Thus the invention may be applied to stem cell lines including adult, fetal, embryonic, neonatal or juvenile stem cell lines. Stem cell lines may be clonal i.e. they may have originated from a single stem cell. In one embodiment, the invention may be applied to existing stem cell lines, particularly to existing embryonic and fetal stem cell lines. In other cases the invention may be applied to a newly established stem cell line.

Stem cells may be an existing stem cell line. Examples of existing stem cell lines which may be used in the invention include the human embryonic stem cell line provided by Geron (Menlo Park, Calif.) and the neural stem cell line provided by ReNeuron (Guildford, United Kingdom). In some embodiments, the stem cell line may be one which is a freely available stem cell, access to which is open. Additional sources for stem cell lines include but are not limited to BresaGen Inc. of Australia; CyThera Inc.; the Karolinska Institute of Stockholm, Sweden; Monash University of Melbourne, Australia; National Centre for Biological Sciences of Bangalore, India; Reliance Life Sciences of Mumbai, India; Technion-Israel Institute of Technology of Haifa, Israel; the University of California at San Francisco; Goteborg University of Goteborg, Sweden; and the Wisconsin Alumni Research Foundation.

Reference herein to stem cell generally includes the embodiment mentioned also being applicable to stem cell lines unless, for example, it is evident that target cells are freshly isolated stem cells or stem cells are resident stem cells in vivo. The invention is applicable to freshly isolated stem cells and also to cell populations comprising stem cells. The invention may also be used to control the differentiation of stem cells in vivo.

An initial step in the methods of the invention may be the isolation of suitable stem cells. Methods for isolating particular types of stem cells are well known in the art and may be used to obtain stem cells for use in the invention. The methods may, for example, be used to recover stem cells from intended recipients of medicaments of the invention. Cell surface markers characteristic of stem cells may be used to isolate the stem cells, for example, by cell sorting. Stem cells may be obtained from any of the types of subjects mentioned herein and in particular from those suffering from any of the disorders mentioned herein.

In some embodiments stems cells may be obtained by using the methods of the invention to reverse the differentiation of differentiated cells to give stem cells. In particular, differentiated cells may be recovered from a subject, treated in vitro in order to produce stem cells, the stem cells obtained may then be manipulated as desired and differentiated before (and/or after) return to the subject. As stem cells typically represent a very small minority of the cells present in an individual such an approach may be preferable. It may also mean that stem cells are more easily derivable from specific individuals and may eliminate the need for embryonic stem cells. In addition, typically such an approach will be less labor intensive and expensive than methods for isolating stem cells themselves. In some cases, stem cells may be isolated from a subject, differentiated in vitro and then returned to the same subject.

In many embodiments stem cells may be any of the types of stem cells mentioned herein and may be in any of the organisms mentioned herein. Target stem cells may be present in any of the organs, tissues or cell populations of the body in which stem cells exist, including any of those mentioned herein. Target stem cells will typically be resident stem cells naturally occurring in the subject, but in some cases stem cells produced using the methods of the invention may be transferred into the subject and then induced to differentiate by transfer of RNA.

Various techniques for isolating, maintaining, expanding, characterizing and manipulating stem cells in culture are known and may be employed. In some cases genetic modifications may be introduced into genomes of stem cells. Stem cells lend themselves to such manipulation as clonal lines can be established and readily screened using techniques such as PCR or Southern blotting.

In some instances stem cells may originate from an individual or animal with a genetic defect. Methods described herein may be used to make modifications to correct or ameliorate the defect. For example, a functional copy of a missing or defective gene may be introduced into the genome of the cell. In a particular embodiment, differentiated cells may be obtained from an individual with a genetic defect, stem cells obtained from the differentiated cells using the methods disclosed herein, the genetic defect corrected or ameliorated and then either the stem cells or differentiated cells obtained from them will be used for treating the original subject or in the manufacture of medicaments for treating the original subject.

### Overview

The present invention relates to methods for the genetic modification of cells for example stem cells by the use of engineered recombination sites which allow the stable insertion of nucleic acid molecules such as complex expression vectors. Stem cells used for the invention may be embryonic stem cells, adult stem cells or progenitor cells. When embryonic stem cells are used it is possible to produce a transgenic animal from embryonic stem cells in which all of the animals' stem cells contain the engineered recombination site. In such an animal, adult stem cells can be harvested and engineered recombination sites used to insert nucleic acid molecules such as complex expression vectors. Alternatively, the expression vector can be inserted into the stem cell before the transgenic animal is produced so that the expression vector is present throughout embryonic development. The ability to create genetically engineered stem cells allows for the study of effects of drug compounds on cell fate, protein-protein interactions, and the activity of specific cell signaling pathways in the context of normal cellular environments. Whole animal models that may be generated with this platform technology may enable therapeutic studies, drug toxicity testing, and stem cell transplant tracking using fluorescent proteins and MRI contrasting reporters. In some embodiments, the use of the invention will allow creation of adult stem and progenitor cell populations pre-engineered with reporters and/or perturbation reagent combinations or ready-engineered populations (using an existing specific integrase target site) for genomic manipulation at very early passage numbers. Such ready-engineering may permit genetic manipulation in non immortal adult stem cells which has been impossible so far. In cases where adult stem cells are used, expression vectors may contain genes that correct genetic errors so that modified stem cells may be returned to the animal as a form of treatment for a particular medical condition.

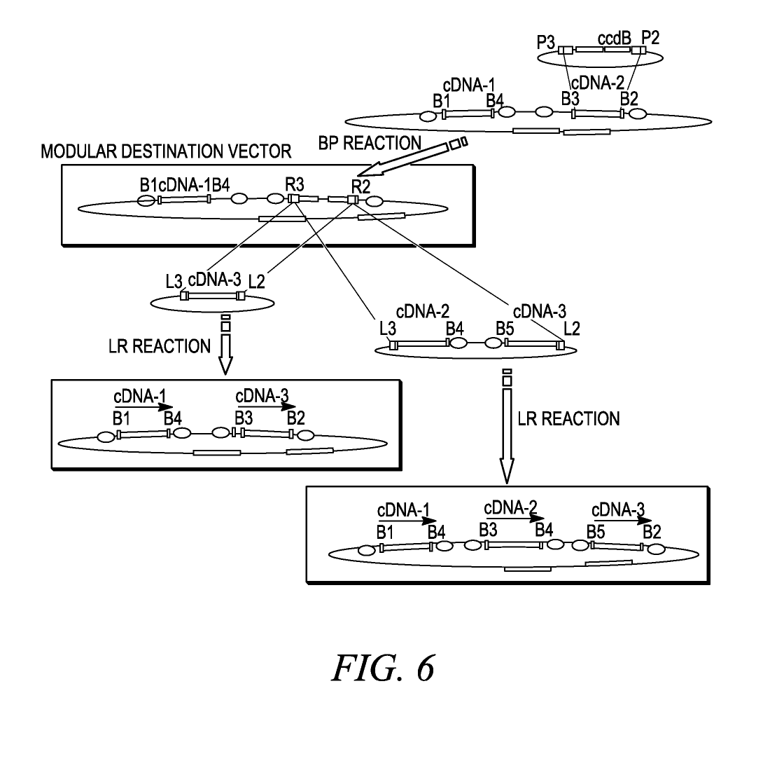

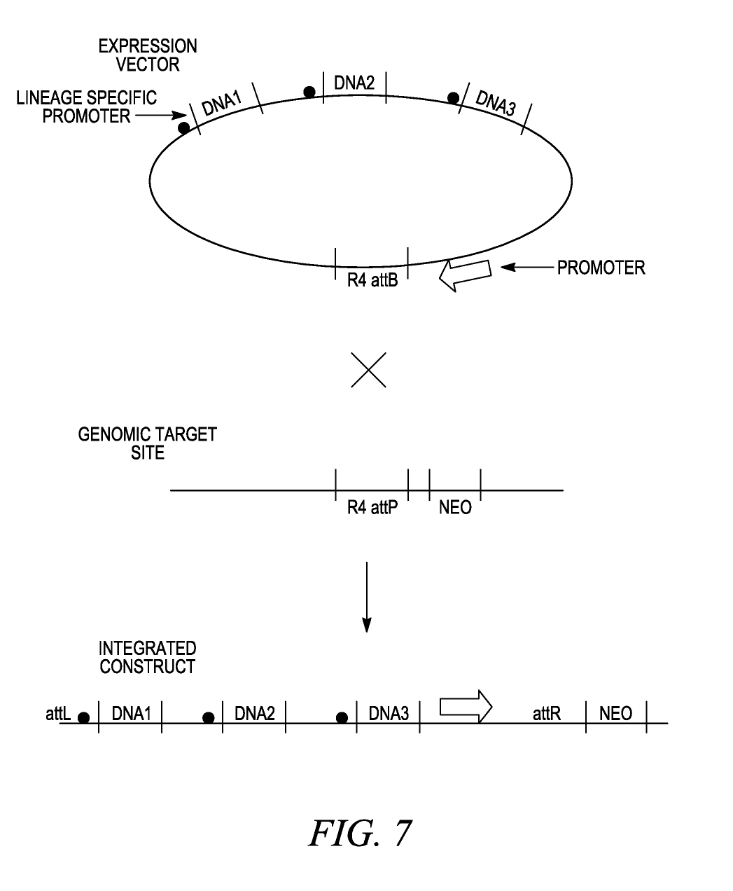

In order to allow selection of stem cells in which the expression vectors have been stably integrated, target stem cells may be engineered to contain an antibiotic resistance gene or other selectable marker that is not operably linked to a promoter. The transfected expression vector may comprise a promoter positioned so that when successfully integrated, it regulates the expression of the selectable marker. A non-limiting example of this selection scheme is illustrated in FIG. 6. The incoming expression vector may be constructed by the use of site-specific recombinational cloning techniques which allow the construction of complex vectors with large numbers of genetic elements arranged in a specific order.

Stem cells can be maintained in a desired state of differentiation by the use of differentiation state or cell lineage associated promoters that are operably linked to an antibiotic resistance gene. A differentiation state associated promoter is one in which the function of the promoter is tied to the differentiation state of the cell. When the cell begins to differentiate, the function of the promoter decreases and the expression of linked antibiotic resistance gene is reduced and the cell becomes susceptible to the appropriate antibiotic. A cell lineage associated promoter is one in which the promoter displays differential activity in a specific cell lineage. A cell lineage associated promoter may not be functional or will have different activity in cells of a different lineage. This same principal can be used to select stem cells that move down a particular differentiation pathway where an antibiotic resistance gene is operably linked to a promoter which becomes active only when the stem cell differentiates along the desired lineage pathway. The appropriate antibiotic can then be used to eliminate cells which have differentiated down the wrong pathway or which belong to the wrong lineage.

In some embodiments stem cells will be engineered to contain multiple differentiation state or lineage associated promoters each operably linked to a unique antibiotic resistance gene. This allows selection stem cells that have a variety of antibiotic resistance profiles depending on the differentiation pathway they follow. In some instances all of the promoters may remain transcriptionally active so that the stem cells will remain resistant to all of the antibiotics. In other instances, some promoters may remain or become transcriptionally active in one differentiation pathway but not in another pathway. This will result in specific patterns of antibiotic resistance for specific differentiation pathways and allow for specifically selecting stem cells which follow desired differentiation pathway.

The invention disclosed herein comprises a method of specifically modifying a genome of a stem cell. The method of the invention is based, in part, on the discovery that there exist in various genomes specific nucleic acid sequences, herein called pseudo sites, that may be distinct from wild-type recombination sequences and that can be recognized by a site-specific recombinase and used to promote the insertion of heterologous genes or polynucleotides into the genome.

### Recombinases

Two major families of site-specific recombinases from bacteria and unicellular yeasts have been described: the integrase family includes Cre, Flp, R, and λ integrase (Argos, et al., EMBO J. 5:433-440, (1986)) and the resolvase/invertase family includes some phage integrases, such as, those of phages phiC31, R4, and TP-901 (Hallet and Sherratt, FEMS Microbiol. Rev. 21:157-178, (1997)). While not wishing to be bound by descriptions of mechanisms, strand exchange catalyzed by site specific recombinases typically occurs in two steps of (1) cleavage and (2) rejoining involving a covalent protein-DNA intermediate formed between the recombinase enzyme and the DNA strand(s).

The nature of the catalytic amino acid residue of the recombinase enzyme and the line of entry of the nucleophile can be different for the two recombinase families For cleavage catalyzed by the invertase/resolvase family, for example, the nucleophile hydroxyl is derived from a serine and the leaving group is the 3′-OH of the deoxyribose. For the integrase family, the catalytic residue is, for example, a tyrosine and the leaving group is the 5′-OH. In both recombinase families, the rejoining step is the reverse of the cleavage step. Recombinases particularly useful in the practice of the invention are those that function in a wide variety of cell types, in part because they do not require any host specific factors. Suitable recombinases include Cre, Flp, R, and the integrases of phages phiC31, TP901-1, R4, and the like. Some characteristics of the two recombinase families are discussed below.

### Cre-Like Recombinases

The recombinase activity of Cre has been studied as a model system for the integrases. Cre is a 38 kD protein isolated from bacteriophage P1. It catalyzes recombination at a 34 basepair stretch of DNA called loxP. The loxP site has the sequence 5′-ATAACTTCGTATA GCATACAT TATACGAAGTTAT-3′ (SEQ ID NO:1) consisting of two thirteen basepair palindromic repeats flanking an eight basepair core sequence. The repeat sequences act as Cre binding sites with the crossover point occurring in the core. Each repeat appears to bind one protein molecule wherein the DNA substrate (one strand) is cleaved and a protein DNA intermediate is formed having a 3′-phosphotyrosine linkage between Cre and the cleaved DNA strand. Crystallography and other studies suggest that four proteins and two loxP sites form a synapsed structure in which the DNA resembles models of four-way Holliday-junction intermediates, followed by the exchange of a second set of strands to resolve the intermediate into recombinant products (see, Guo, et al, Nature 389:40-46, (1997)). The asymmetry of the core region is responsible for directionality of the recombination reaction. If the two recombination sites are repeated in the same orientation, the outcome of strand exchange is integration or excision. If the two sites are placed in the opposite orientation, the outcome is inversion of the sequence between the two sites (Yang and Mizuuchi, Structure 5:1401-1406, (1997)).

Cre has been shown to be active in a wide variety of cellular backgrounds including yeast (Sauer, Mol. Cell. Biol. 7:2087-2096, (1987)), plants (Albert, et al, Plant J. 7:649-659, (1995); Dale and Ow, Gene 91:79-8S, (1990); Odell, et al, Mol. Gen. Genet. 223:369-378, (1990)) and mammals, including both rodent and human cells (van Deursen, et al, Proc. Natl. Acad. Sci. USA 92:7376-7380, (1995); Agah, et al, J. Clin. Invest. 100:169-179, (1997); Baubonis, and Sauer, 21:2025-2029, (1993); Sauer and Henderson, New Biologist 2:441-449, (1990)). As the loxP site is known only to occur in the P1 phage genome, use of the enzyme in other cell types requires the prior insertion of a loxP site into the genome, which using currently available technologies is generally a low-frequency and random event with all of the drawbacks inherent in such a procedure. The loxP site can be targeted to a specific location by using homologous recombination, but, again, that process occurs at a very low frequency.

Several studies have suggested the possibility that an exact match of the loxP sequence is not required for Cre-mediated recombination (Sternberg, et al, J. Mol. Biol. 150:487-507, (1981); Sauer, J. Mol. Biol. 223:911-928, (1992); Sauer, Nucleic Acids Research 24:4608-4613, (1996)). The efficiency of recombination, however, has generally been three to four orders of magnitude less efficient than wild-type loxP. Sauer attempted to identify sequences similar to loxP in the human genome without success (Sauer, Nucleic Acids Research 24:4608-4613, (1996)).

Flp, a recombinase of the integrase family with similar properties to Cre has been identified in strains of Saccharomyces cerevisiae that contain 2μ-circle DNA. Flp recognizes a DNA sequence consisting of two thirteen basepair inverted repeats flanking an eight basepair core sequence (5′-GAAGTTCCTATAC TTCTAGAA GAATAGGAACTTC-3′) (SEQ ID NO:2) called FRT. A third repeat follows at the 3′ end in the natural sequence but does not appear to be required for recombinase activity. Like Cre, Flp is functional in a wide variety of systems including bacteria (Huang, et al, J Bacteriology 179:6076-6083, (1997)), insects (Golic and Lindquist, Cell 59:499-509, (1989); Golic and Golic, Genetics 144:1693-1711, (1996)), plants (Lyznik, et al, Nucleic Acids Res 21:969-975, (1993)) and mammals. These studies have likewise required that a FRT sequence be inserted into the genome to be modified.

A related recombinase, known as R, is encoded by the pSRi plasmid of the yeast Zygosaccharomyces rouxii (Araki, et al., J. Mol. Biol. 182:191-203, (1985), herein incorporated by reference). This recombinase may have properties similar to those described above.

### Resolvase/Integrase Recombinases

Unlike the Cre/λ integrase family of recombinases, members of the resolvase subfamily of recombinase enzymes typically contain an N-terminal catalytic domain having a high degree (>35%) of sequence homology among the subfamily members (Crellin and Rood, J Bacteriology 179:5148-5156, (1997); Christiansen, et al, J. Bacteriology 17:5164-55173, (1996)). Like some of the Cre-type recombinases, however, some resolvases do not require host specific accessory factors (Thorpe and Smith, PNAS USA 95:5505-5510, (1998)).

The process of strand exchange used by the resolvases is somewhat different than the process used by Cre. This process is described but is not intended to be limiting. The resolvases usually make cuts close to the center of the crossover site, and the top and bottom strand cuts are often staggered by 2 basepairs, leaving recessed 5′ ends. A protein-DNA linkage is formed between phosphodiester from the 5′ DNA end and a conserved serine residue close to the amino terminus of the recombinase. As with the Cre-like invertases, two protein units are bound at each crossover site, however, no equivalent to the Holiday junction intermediate is formed (see Stark, et al, Trends in Genetics 8:432-439, (1992), incorporated by reference herein).

The nucleic acid sequences recognized as recombination sites by a subset of the resolvase family, including some phage integrases, differ in several ways from the recombination site recognized by Cre. The sites used for recognition and recombination of the phage and bacterial DNAs (the native host system) are generally non-identical, although they typically have a common core region of nucleic acids. The bacterial sequence is generally called the attB sequence (bacterial attachment) and the phage sequence is called the attP sequence (phage attachment). Because they are different sequences, recombination will result in a stretch of nucleic acids (called attL or attR for left and right) that is neither an attB sequence or an attP sequence, and is probably functionally unrecognizable as a recombination site to the relevant enzyme, thus removing the possibility that the enzyme will catalyze a second recombination reaction that would reverse the first.

The individual resolvases and the nucleic acid sequences that they recognize have been less well characterized than Cre and Flp, although many of the core sequences have been identified. The core sequences of some of the resolvases useful in the practice of the invention can include, without limitation, the following sequences: phiC31-5′-TTG; TP901-1-5′-TCAAT; and R4-5′-GAAGCAGTGGTA (SEQ ID NO:3). (See Rausch and Lehmann, NAR 19:5187-5189, (1991); Shirai, et al, J Bacteriology 173:4237-4239, (1991); Crellin and Rood, J Bacteriology 179:5148-5156, (1997); Christiansen, et al, J. Bacteriology 176:1069-1076, (1994); Brondsted and Hammer, Applied & Environmental Microbiology 65:752-758, (1999); all of which are incorporated by reference herein.)

### Recombination Sites

There are native recombination sites in the genomes of a variety of organisms, where the native recombination site does not necessarily have a nucleotide sequence identical to the wild-type recombination sequences (for a given recombinase). Such native recombination sites are nonetheless sufficient to promote recombination meditated by the recombinase. Such recombination site sequences are referred to herein as “pseudo-recombination sequences.” For a given recombinase, a pseudo-recombination sequence may be functionally equivalent to a wild-type recombination sequence (generally react with lower efficiency), may occur in an organism other than that in which the recombinase is found in nature, and may have sequence variation relative to the wild type recombination sequences.

In the practice of the present invention, wild-type recombination sites, pseudo-recombination sites, and hybrid-recombination sites can be used in a variety of ways in the construction of targeting vectors. Following here are non-limiting examples of how these sites may be employed in the practice of the present invention.

In one embodiment of the present invention, the recombinase (for example, phiC31) recognizes a recombination site where sequence of the 5′ region of the recombination site can differ from the sequence of the 3′ region of the recombination sequence. For example, for the phage phiC31 attP (the phage attachment site), the core region is 5′-TTG-3′ the flanking sequences on either side are represented here as attP5′ and attP3′, the structure of the attP recombination site is, accordingly, attP5′-TTG-attP3′. Correspondingly, for the native bacterial genomic target site (attB) the core region is 5′-TTG-3′, and the flanking sequences on either side are represented here as attB5′ and attB3′, the structure of the attB recombination site is, accordingly, attB5′-TTG-attB3′. After a single-site, phiC31 integrase mediated, recombination event takes place the result is the following recombination product: attB5′-TTG-attP3′{phiC31 vector sequences}attP5′-TTG-attB3′. Typically, after recombination the post-recombination recombination sites are no longer able to act as substrate for the phiC31 recombinase. This results in stable integration with little or no recombinase mediated excision.

In this aspect, when selecting pseudo-recombination sites in a target stem cell, the genomic sequences of the target stem cell can be searched for suitable pseudo-recombination sites using either the attP or attB sequences associated with a particular recombinase. Functional sizes and the amount of heterogeneity that can be tolerated in these recombination sequences can be evaluated.

When a pseudo-recombination site is identified using either attP or attB search sequences, the other recombination site can be used in the targeting construct. For example, if attP for a selected recombinase is used to identify a pseudo-recombination site in the target stem cell genome, then the wild-type attB sequence can be used in the targeting construct. In an alternative example, if attB for a selected recombinase is used to identify a pseudo-recombination site in the target stem cell genome, then the wild-type attP sequence can be used in the expression construct.

In further embodiments of the invention the genomic location of pseudo sites can be determined. Stem cells may be transfected with a plasmid comprising a first recombination site, a second wild type recombination site, a first selectable marker and a second conditional selectable marker. Stem cells in which the plasmid has been successfully integrated are selected for by use of the first selectable marker. The site of integration of the plasmid and vector can then be determined by rescuing the plasmid and sequencing it. The rescued plasmid may contain stem cell derived sequences at its' ends. These sequences can be used with publicly available databases to determine the exact genomic location of the plasmid integration site.

Plasmid rescue may be performed by isolating total genomic DNA and digesting with one or more restriction enzymes that preferably cut outside of the integrated plasmid sequence. In some embodiments the restriction enzymes chosen produce sticky ends. After restriction, DNA fragments may be circularized in a ligation reaction and DNA then transformed into a competent E. coli cell such as DH10B or TOP10 cells (Invitrogen Corp., Carlsbad, Calif.). DNA may then be isolated from drug resistant colonies and the presence of plasmid sequences confirmed by restriction analysis. The rescued plasmid DNA may then be sequenced by standard methods. Genome derived sequences from the ends of the rescued plasmid may then be compared to databases to locate the exact site of integration into the genome.

A vector comprising a developmental promoter operably linked to a reporter and a recombination site complementary to the second wild type recombination site of the plasmid may be transfected into the stem cell along with a recombinase specific for the second wild type recombination site such that the vector is integrated into the genome. The promoter of the vector may be located such that when inserted into the genome by the recombination reaction it becomes operably linked to the second conditional selectable marker of the plasmid. Stem cells with successfully integrated vectors can be selected for using the selective agent associated with the second conditional marker.

Expression vectors contemplated by the invention may contain additional nucleic acid fragments such as control sequences, marker sequences, selection sequences and the like as discussed below.

### Expression Vectors and Methods of the Present Invention

The present invention also provides means for targeted insertion of a polynucleotide (or nucleic acid sequence(s)) of interest into a stem cell genome by, for example, (i) providing a recombinase, wherein the recombinase is capable of facilitating recombination between a first recombination site and a second recombination site, (ii) providing an expression construct having a first recombination sequence and a polynucleotide of interest, (iii) introducing the recombinase, mRNA encoding the recombinase or a vector expressing the recombinase and the expression vector into a cell which contains in its nucleic acid the second recombination site, wherein said introducing is done under conditions that allow the recombinase to facilitate a recombination event between the first and second recombination sites.

In one aspect of the present invention, at least one pseudo-recombination site for a selected recombinase may be identified in a target stem cell of interest. These sites can be identified by several methods including searching all known sequences derived from the cell of interest against a wild-type recombination site (e.g., attB or attP) for a selected recombinase. The functionality of pseudo-recombination sites identified in this way can then be empirically evaluated following the teachings of the present specification to determine their ability to participate in a recombinase-mediated recombination event.

### Expression Vectors

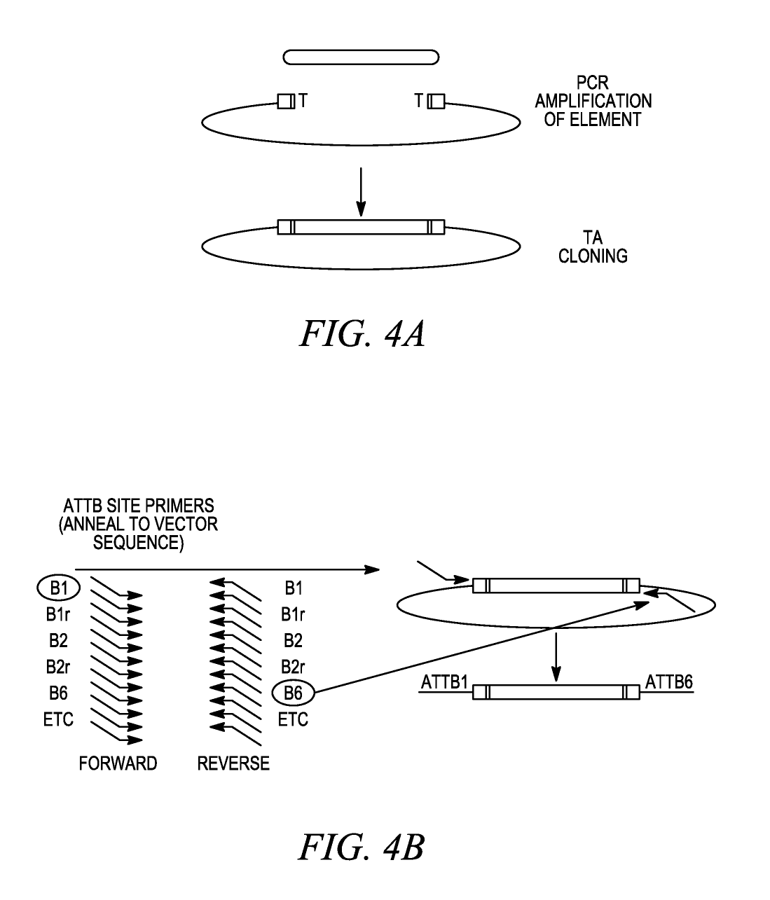

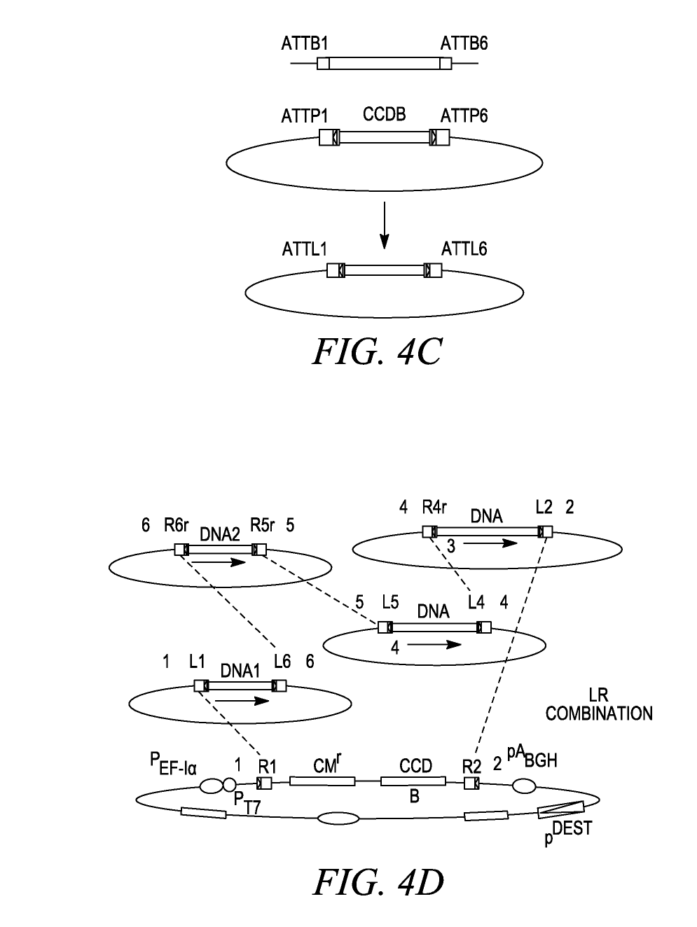

In many embodiments of the present invention, a collection of useful genetic elements or a genetic toolbox is created. Components of the toolbox may comprise transcriptional promotors and reporters. Suitable promoters include, but are not limited to, constitutive viral, human and mouse tissue-specific, regulatable promoters. Suitable reporters include, but are not limited to, green fluorescent protein (GFP) variants, β-lactamase, lumio, magnetic resonance imaging (MRI), and positron emission tomography (PET) contrasting proteins. Additional components of the toolbox could include other elements useful for genomic engineering such as toxin genes, recombination sites, internal ribosomal entry segment (IRES) sequences, etc. An outline of one embodiment of a method for assembling expression vectors for use in the present invention is shown in FIGS. 4a-4d.

The elements of the toolbox may first be placed into entry clones. The first step of preparing an entry clone may be to amplify the genetic element by polymerase chain reaction (PCR) followed by cloning into a TA or any other cloning vector (FIG. 4a). General procedures for PCR are taught in MacPherson et al., PCR: A Practical Approach, (IRL Press at Oxford University Press, (1991)). PCR conditions for each application reaction may be empirically determined. A number of parameters influence the success of a reaction. Among these parameters are annealing temperature and time, extension time, Mg²⁺ and ATP concentration, pH, and the relative concentration of primers, templates and deoxyribonucleotides. After amplification, the resulting fragments can be detected by agarose gel electrophoresis followed by visualization with ethidium bromide staining and ultraviolet illumination.

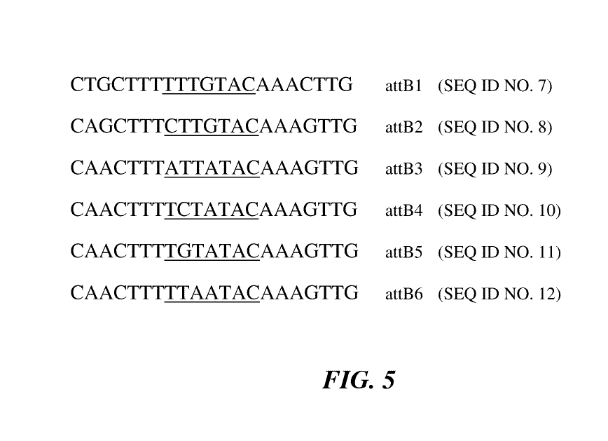

The TA Cloning® Kit from Invitrogen (catalog No. KNM2000-01, Carlsbad, Calif.) provides suitable reagents for the TA cloning reaction. Sequences which may not be adequately amplified by PCR can be prepared synthetically using methods well known in the art. Specific modified attB sites may then be added to the cloned element. The modified attB sites provide an ‘address’ for each element to ensure that each entry clone is in the proper order and orientation in the destination vector. Non-limiting examples of modified attB sites are shown in FIG. 5. The addition of selected modified attB sites to the entry clone is illustrated (FIG. 4b). The modified attB sites may be added in a PCR reaction using primers which universally anneal with the vectors used in the cloning reaction and that contain the modified attB sequence. The product of this PCR reaction may be recombined with a vector containing a toxic gene such as ccdB flanked by modified attP sites designed to recombine with the modified attB sites of the PCR product. The PCR product exchanges with the toxic gene during the recombination reaction and the loss of the toxic gene can be used to select for the vectors that have been successfully recombined (FIG. 4c). This cloned PCR product is an entry clone containing a genetic element flanked by attL and/or attR sites.

The final expression vector is produced by recombining entry clones containing the desired genetic elements with a destination vector containing appropriate attR sites and a selection marker (FIG. 4d). This procedure can be used to produce a simple expression vector with for example two elements, a promoter and a gene to be expressed, or more complex expression vectors with, three, four, five, seven, ten, twelve, fifteen, twenty, thirty, fifty, seventy-five, one hundred, two hundred, etc. genetic elements. Intermediate destination vectors may be used prepare expression vectors with large numbers of genetic elements as outlined in FIG. 6.

The number of genes which may be connected in using methods of the invention in a single step will in general be limited by the number of recombination sites with different specificities which can be used. Further, recombination sites can be chosen so as to link nucleic acid segments in one reaction and not engage recombination in later reactions. For example a series of concatamers of ordered nucleic acid segments can be prepared using attL and attR sites and LR Clonase™. These concatamers can then be connected to each other and, optionally, other nucleic acid molecules using another LR reaction. Numerous variations of this process are possible.

A variety of expression vectors are suitable for use in the practice of the present invention. In general, an expression vector will have one or more of the following features: a promoter, promoter-enhancer sequences, a selection marker sequence, an origin of replication, an inducible element sequence, an epitope-tag sequence, and the like.

Promoter and promoter-enhancer sequences are DNA sequences to which RNA polymerase binds and initiates transcription. The promoter determines the polarity of the transcript by specifying which strand will be transcribed. Most promoters utilized in expression vectors are transcribed by RNA polymerase II. General transcription factors (GTFS) first bind specific sequences near the start and then recruit the binding of RNA polymerase II. In addition to these minimal promoter elements, small sequence elements are recognized specifically by modular DNA-binding/trans-activating proteins (e.g. AP-1, SP-1) that regulate the activity of a given promoter. Viral promoters serve the same function as eukaryotic promoters and either provide a specific RNA polymerase in trans (bacteriophage T7) or recruit cellular factors and RNA polymerase (SV40, RSV, CMV). Viral promoters are one example, as they are generally particularly strong promoters.

Promoters may be, furthermore, either constitutive or regulatable (i.e., inducible or derepressible). Inducible elements are DNA sequence elements which act in conjunction with promoters and bind either repressors (e.g. lacO/LAC Iq repressor system in E. coli) or inducers (e.g. gal1/GAL4 inducer system in yeast). In either case, transcription is virtually “shut off” until the promoter is derepressed or induced, at which point transcription is “turned-on.”

Exemplary eukaryotic promoters include, but are not limited to, the following: the promoter of the mouse metallothionein I gene sequence (Hamer et al., J. Mol. Appl. Gen. 1:273-288, (1982)); the TK promoter of Herpes virus (McKnight, Cell 31:355-365, (1982)); the SV40 early promoter (Benoist et al., Nature (London) 290:304-310, (1981)); the yeast gall gene sequence promoter (Johnston et al., Proc. Natl. Acad. Sci. (USA) 79:6971-6975, (1982)); Silver et al., Proc. Natl. Acad. Sci. (USA) 81:5951-59SS, (1984)), the CMV promoter, the EF-1 promoter, Ecdysone-responsive promoter(s), tetracycline-responsive promoter, and the like.

Exemplary promoters for use in the present invention are selected such that they are functional in the cell type (and/or animal or plant) into which they are being introduced.

Selection markers are valuable elements in expression vectors as they provide a means to select for growth of only those stem cells that contain a vector. Such markers are of two types: drug resistance and auxotrophic. A drug resistance marker enables cells to detoxify an exogenously added drug that would otherwise kill the cell. Auxotrophic markers allow cells to synthesize an essential component (usually an amino acid) while grown in media that lacks that essential component.

Common selectable marker genes include those for resistance to antibiotics such as ampicillin, tetracycline, kanamycin, bleomycin, streptomycin, hygromycin, neomycin, Zeocin™, and the like. Selectable auxotrophic genes include, for example, hisD, that allows growth in histidine free media in the presence of histidinol.

A further element useful in an expression vector is an origin of replication. Replication origins are unique DNA segments that contain multiple short repeated sequences that are recognized by multimeric origin-binding proteins and that play a key role in assembling DNA replication enzymes at the origin site. Suitable origins of replication for use in expression vectors employed herein include E. coli oriC, colE1 plasmid origin, 2μ and ARS (both useful in yeast systems), sf1, SV40, EBV oriP (useful in mammalian systems), and the like.

Epitope tags are short peptide sequences that are recognized by epitope specific antibodies. A fusion protein comprising a recombinant protein and an epitope tag can be simply and easily purified using an antibody bound to a chromatography resin. The presence of the epitope tag furthermore allows the recombinant protein to be detected in subsequent assays, such as Western blots, without having to produce an antibody specific for the recombinant protein itself. Examples of commonly used epitope tags include V5, glutathione-S-transferase (GST), hemaglutinin (HA), the peptide Phe-His-His-Thr-Thr, chitin binding domain, and the like.

A further useful element in an expression vector is a multiple cloning site or polylinker. Synthetic DNA encoding a series of restriction endonuclease recognition sites is inserted into a plasmid vector, for example, downstream of the promoter element. These sites are engineered for convenient cloning of DNA into the vector at a specific position.

The foregoing elements can be combined to produce expression vectors suitable for use in the methods of the invention. Those of skill in the art would be able to select and combine the elements suitable for use in their particular system in view of the teachings of the present specification.

Individual elements of the genetic toolbox including but not limited to cloned genetic elements, entry clones containing individual genetic elements, destination vectors, recombinases and recombinase-coding sequences of the present invention can be formulated into kits. Components of such kits can include, but are not limited to, containers, instructions, solutions, buffers, disposables, and hardware.

### Stem Cells

Stem cells suitable for modification employing the methods of the invention include but are not limited to those stem cell's whose genome contains an homologous recombination site or a pseudo-recombination sequence.

In addition, plant stem cells are also available as hosts, and control sequences compatible with plant cells are available, such as the cauliflower mosaic virus ³⁵S and 19S, nopaline synthase promoter and polyadenylation signal sequences, and the like. Appropriate transgenic plant cells can be used to produce transgenic plants.

In representative embodiments, to allow the controlled introduction of the expression vector into the genome of the stem cell, a wild type R4 integration site is introduced into the stem cell. To control the site of integration of the R4 site, the R4 containing vector will have a sequence that will allow it to recombine with a phiC31 pseudo attP site or a homologous recombination site. In embodiments where a pseudo attP site is used, a phiC31 integrase expression vector will be transfected along with the R4 vector.

Other methods of introducing recombinase or integrase activity may be used with the present invention. Methods of introducing functional proteins into cells are well known in the art. Introduction of purified recombinase protein ensures a transient presence of the protein and its function, which is one embodiment. Alternatively, a gene encoding the recombinase can be included in an expression vector used to transform the cell. In many embodiments, the recombinase is present for only such time as is necessary for insertion of the nucleic acid fragments into the genome being modified. Thus, the lack of permanence associated with most expression vectors is not expected to be detrimental.

The recombinases used in the practice of the present invention can be introduced into a target cell before, concurrently with, or after the introduction of a targeting vector. The recombinase can be directly introduced into a cell as a protein, for example, using liposomes, coated particles, or microinjection. Alternately, a polynucleotide encoding the recombinase can be introduced into the cell using a suitable expression vector. The targeting vector components described above are useful in the construction of expression cassettes containing sequences encoding a recombinase of interest. Expression of the recombinase is typically desired to be transient. Accordingly, vectors providing transient expression of the recombinase are use in some embodiments of the present invention. However, expression of the recombinase can be regulated in other ways, for example, by placing the expression of the recombinase under the control of a regulatable promoter (i.e., a promoter whose expression can be selectively induced or repressed). Further, recombinase can be delivered to the cell via transfection with recombinase protein or mRNA.

Sequences encoding recombinases useful in the practice of the present invention are known and include, but are not limited to, the following: Cre, Sternberg, et al., J. Mol. Biol. 187:197-212; phiC31, Kuhstoss and Rao, J. Mol. Biol. 222:897-908, (1991); TP901-1, Christiansen, et al., J. Bact. 178:5164-5173, (1996); R4, Matsuura, et al., J. Bact. 178:3374-3376, (1996).

Recombinases for use in the practice of the present invention can be produced recombinantly or purified using techniques well known in the art. Polypeptides having the desired recombinase activity can be purified to a desired degree of purity by methods known in the art of protein purification including, but not limited to, ammonium sulfate precipitation, size fractionation, affinity chromatography, HPLC, ion exchange chromatography, heparin agarose affinity chromatography (e.g., Thorpe & Smith, Proc. Nat. Acad. Sci. 95:5505-5510, (1998).)

Stem cells modified by the methods of the present invention can be maintained under conditions that, for example, (i) keep them alive but do not promote growth, (ii) promote growth of the cells, and/or (iii) cause the cells to differentiate or dedifferentiate. Cell culture conditions are typically permissive for the action of the recombinase in the cells, although regulation of the activity of the recombinase may also be modulated by culture conditions (e.g., raising or lowering the temperature at which the cells are cultured). For a given cell, cell-type, tissue, or organism, culture conditions are known in the art. These conditions include but are not limited to the use of defined media and matrices for the maintenance of stem cells in culture.

### Transgenic Plants and Non-Human Animals

In another embodiment, the present invention comprises transgenic plants and nonhuman transgenic animals whose genomes have been modified by employing the methods and compositions of the invention. Transgenic animals may be produced employing the methods of the present invention to serve as a model system for the study of various disorders and for screening of drugs that modulate such disorders.

A “transgenic” plant or animal refers to a genetically engineered plant or animal, or offspring of genetically engineered plants or animals. A transgenic plant or animal usually contains material from at least one unrelated organism, such as, from a virus. The term “animal” as used in the context of transgenic organisms means all species except human. It also includes an individual animal in all stages of development, including embryonic and fetal stages. Farm animals (e.g., chickens, pigs, goats, sheep, cows, horses, rabbits and the like), rodents (such as mice), and domestic pets (e.g., cats and dogs) are included within the scope of the present invention. In some embodiments, the animal is a mouse or a rat.

The term “chimeric” plant or animal is used to refer to plants or animals in which the heterologous gene is found, or in which the heterologous gene is expressed in some but not all cells of the plant or animal.

The term transgenic animal also includes a germ cell line transgenic animal. A “germ cell line transgenic animal” is a transgenic animal in which the genetic information provided by the invention method has been taken up and incorporated into a germ line cell, therefore conferring the ability to transfer the information to offspring. If such offspring, in fact, possess some or all of that information, then they, too, are transgenic animals.

Methods of generating transgenic plants and animals are known in the art and can be used in combination with the teachings of the present application.

In one embodiment, a transgenic animal of the present invention is produced by introducing into a single cell embryo a nucleic acid construct, comprising a phiC31 recombination site capable of recombining with a pseudo att site found within the genome of the organism from which the cell was derived and a nucleic acid fragment comprising a R4 integration site, in a manner such that the R4 integration site is stably integrated into the DNA of germ line cells of the mature animal and is inherited in normal Mendelian fashion. In other embodiments an R4 site is used to stably integrate a phiC31 integration site into the genome of the animal. In further embodiments a selection marker is integrated into the genome of the animal along with the integration site so that successful events can be selected for.

By way of example only, to prepare a transgenic mouse, female mice are induced to superovulate. After being allowed to mate, the females are sacrificed by CO₂ asphyxiation or cervical dislocation and embryos are recovered from excised oviducts. Surrounding cumulus cells are removed. Pronuclear embryos are then washed and stored until the time of injection. Randomly cycling adult female mice are paired with vasectomized males. Recipient females are mated at the same time as donor females. Embryos then are transferred surgically. The procedure for generating transgenic rats is similar to that of mice. See Hammer, et al., Cell 63:1099-1112, (1990)). Rodents suitable for transgenic experiments can be obtained from standard commercial sources such as Charles River (Wilmington, Mass.), Taconic (Germantown, N.Y.), Harlan Sprague Dawley (Indianapolis, Ind.), etc.

The procedures for manipulation of the rodent embryo and for microinjection of DNA into the pronucleus of the zygote are well known to those of ordinary skill in the art (Hogan, et al., supra). Microinjection procedures for fish, amphibian eggs and birds are detailed in Houdebine and Chourrout, Experientia 47:897-905, (1991)). Other procedures for introduction of DNA into tissues of animals are described in U.S. Pat. No. 4,945,050 (Sandford et al., Jul. 30, (1990)).

Pluripotent or multipotent stem cells derived from the inner cell mass of the embryo and stabilized in culture can be manipulated in culture to incorporate nucleic acid sequences employing invention methods. A transgenic animal can be produced from such cells through injection into a blastocyst that is then implanted into a foster mother and allowed to come to term.

Methods for the culturing of stem cells and the subsequent production of transgenic animals by the introduction of DNA into stem cells using methods such as electroporation, calcium phosphate/DNA precipitation, microinjection, liposome fusion, retroviral infection, and the like are also are well known to those of ordinary skill in the art. See, for example, Teratocarcinomas and Embryonic Stem Cells, A Practical Approach, E. J. Robertson, ed., IRL Press, 1987). Reviews of standard laboratory procedures for microinjection of heterologous DNAs into mammalian (mouse, pig, rabbit, sheep, goat, cow) fertilized ova include: Hogan et al., Manipulating the Mouse Embryo (Cold Spring Harbor Press 1986); Krimpenfort et al., (1991), Bio/Technology 9:86; Palmiter et al., (1985), Cell 41:343; Kraemer et al., Genetic Manipulation of the Early Mammalian Embryo (Cold Spring Harbor Laboratory Press 1985); Hammer et al., (1985), Nature, 315:680; Purcel et al., (1986), Science, 244:1281; Wagner et al., U.S. Pat. No. 5,175,385; Krimpenfort et al., U.S. Pat. No. 5,175,384, the respective contents of which are incorporated by reference.

One embodiment of the procedure is to inject targeted embryonic stem cells into blastocysts and to transfer the blastocysts into pseudopregnant females. The resulting chimeric animals are bred and the offspring are analyzed by Southern blotting to identify individuals that carry the transgene. Procedures for the production of non-rodent mammals and other animals have been discussed by others (see Houdebine and Chourrout, supra; Purcel, et al., Science 244:1281-1288, (1989); and Simms, et al., Bio/Technology 6:179-183, (1988)). Animals carrying the transgene can be identified by methods well known in the art, e.g., by dot blotting or Southern blotting.

The term transgenic as used herein additionally includes any organism whose genome has been altered by in vitro manipulation of the early embryo or fertilized egg or by any transgenic technology to induce a specific gene knockout. The term “gene knockout” as used herein, refers to the targeted disruption of a gene in vivo with loss of function that has been achieved by use of the invention vector. In one embodiment, transgenic animals having gene knockouts are those in which the target gene has been rendered nonfunctional by an insertion targeted to the gene to be rendered non-functional by targeting a pseudo-recombination site located within the gene sequence.

### Gene Therapy and Disorders

A further embodiment of the invention comprises a method of treating a disorder in a subject in need of such treatment. In one embodiment of the method, a stem cell of the subject has a pseudo att sequence. This stem cell is transformed with a nucleic acid construct comprising a wild type phage integration sequence such as phiC31 or R4 and a selection marker. A recombinase is introduced into the stem cell under conditions such that the phage integration sequence is stably inserted into the genome by a recombination event. An expression vector containing one or more genes related to treatment of the condition and a complementary phage integration sequence is then introduced into the cell with the proper recombinase so that the expression vector is stably integrated into the genome of the stem cell. The stem cell is then reintroduced into the subject. Subjects treatable using the methods of the invention include both humans and non-human animals. Such methods utilize the targeting constructs and recombinases of the present invention.

A variety of disorders may be treated by employing the method of the invention including monogenic disorders, infectious diseases, acquired disorders, cancer, and the like. Exemplary monogenic disorders include ADA deficiency, cystic fibrosis, familial-hypercholesterolemia, hemophilia, chronic granulomatous disease, Duchenne muscular dystrophy, Fanconi anemia, sickle-cell anemia, Gaucher's disease, Hunter syndrome, X-linked SCID, and the like.

Infectious diseases treatable by employing the methods of the invention include infection with various types of virus including human T-cell lymphotropic virus, influenza virus, papilloma virus, hepatitis virus, herpes virus, Epstein-Bar virus, immunodeficiency viruses (HIV, and the like), cytomegalovirus, and the like. Also included are infections with other pathogenic organisms such as Mycobacterium Tuberculosis, Mycoplasma pneumoniae, and the like or parasites such as Plasmadium falciparum, and the like.

The term “acquired disorder” as used herein refers to a non-congenital disorder. Such disorders are generally considered more complex than monogenic disorders and may result from inappropriate or unwanted activity of one or more genes. Examples of such disorders include peripheral artery disease, rheumatoid arthritis, coronary artery disease, and the like.

A particular group of acquired disorders treatable by employing the methods of the invention include various cancers, including both solid tumors and hematopoietic cancers such as leukemias and lymphomas. Solid tumors that are treatable utilizing the invention method include carcinomas, sarcomas, osteomas, fibrosarcomas, chondrosarcomas, and the like. Specific cancers include breast cancer, brain cancer, lung cancer (non-small cell and small cell), colon cancer, pancreatic cancer, prostate cancer, gastric cancer, bladder cancer, kidney cancer, head and neck cancer, and the like.

The suitability of the particular place in the genome is dependent in part on the particular disorder being treated. For example, if the disorder is a monogenic disorder and the desired treatment is the addition of a therapeutic nucleic acid encoding a non-mutated form of the nucleic acid thought to be the causative agent of the disorder, a suitable place may be a region of the genome that does not encode any known protein and which allows for a reasonable expression level of the added nucleic acid. Methods of identifying suitable places in the genome are well known in the art.

The expression vector useful in this embodiment is additionally comprised of one or more nucleic acid fragments of interest. Among the nucleic acid fragments of interest for use in this embodiment are therapeutic genes and/or control regions, as previously defined. The choice of nucleic acid sequence will depend on the nature of the disorder to be treated. For example, a nucleic acid construct intended to treat hemophilia B, which is caused by a deficiency of coagulation factor IX, may comprise a nucleic acid fragment encoding functional factor IX. A nucleic acid construct intended to treat obstructive peripheral artery disease may comprise nucleic acid fragments encoding proteins that stimulate the growth of new blood vessels, such as, for example, vascular endothelial growth factor, platelet-derived growth factor, and the like. Those of skill in the art would readily recognize which nucleic acid fragments of interest would be useful in the treatment of a particular disorder.

### Preparation of Target Stem Cells.

A target stem cell is one that has been transfected with a plasmid carrying a recombination site such as an attP or attB site. The presence of these recombination sites allows the easy insertion of expression vectors into the target stem cell. The recombination site can be targeted to a particular locus by any of several means know in the art. These include, but are not limited to, pseudo attP sites, sleeping beauty transposons and homologous recombination. In addition to the integrase-specific site, the plasmid also carries at least a first and second selectable markers. The first selectable marker may in some embodiments be a gene conferring resistance to an antibiotic so that stem cells in which the plasmid has been stably integrated can be selected. Other selection methods known in the art may also be used.

The second selectable marker is used to select for cells which have been stably transformed by an expression vector. The gene which serves as the second selectable marker is positioned in such a way so that it is not under the operable control of a promoter. The incoming expression vector is engineered to contain a promoter that will, upon intergration into the recombination site of the target stem cell, drive expression of the second selectable marker so that stably transformed target stem cells can be selected for.

### Identification of Genes for Bioproduction and Drug Discovery

A reliable approach to identify genes that enhance cell performance like cell viability, productivity, product quality and metabolism of a bioproduction cell line is provided. This is achieved by targeting a plasmid containing a gene of interest into a defined genomic locus in a host cell. An empty vector control or a plasmid containing an unrelated gene may be targeted into the same genomic loci in a parallel experiment. Since all gene constructs are integrated into the same loci, observed phenotypic changes of the host cell can be clearly deduced to the product the gene of interest is coding for. This approach may be used to compare effects of different genes or to screen a library to identify one or more genes that improve one or more cell phenotypes. Once identified, these may be used to engineer a chosen host cell with improved performance. These approaches are generally illustrated in FIGS. 17-19.

Most studies describing enhanced performance of a typical bioproduction cell line use random integration of a plasmid coding for a specific gene and comparing the effects of the gene product on the cell lines to either the parental cell line or a cell line that was generated the same way with a control plasmid. With this approach phenotypic changes can be caused by the experimental conditions and not necessarily by the gene product especially when the parental cell line is used as a control. Some researchers try to circumvent these issues by using inducible expression systems. Cell phenotypes are assessed under non-inducing and inducing conditions. Unfortunately, inducible systems are often leaky and results are inconclusive. Targeted integration of plasmids coding for different genes of interest into the same genomic locus will control for the effects. All cells used for the experiment contain the gene of interest in the same genomic locus and the only difference between these cell lines are the sequences of the inserted genes and the gene product.

The method includes the integration of plasmids into the same genomic loci in separate experiments using the Zinc finger or Endonuclease technology or by homologous in vivo recombination system like the reversible Cre/lox and Flp in or the irreversible system PhiC31 and attR4 integrase system.

The nuclease technologies will require the design and generation of specify Zinc fingers fused to a nuclease domain or an Endonuclease recognizing the targeted genomic DNA sequences. If in vivo recombination systems are used the cell lines are created in two steps. First the bioproduction cell line may be modified by integrating a plasmid with a recombination site (such as for example Lox, Frt or attP site) into the genome. If a reversible system is used the integration copy number may be limited, for example to one. Out of the resulting cell pool containing randomly integrated recombination sites, a clone may be selected and scaled up and banked for subsequent experiments.

Multiple cell lines may be generated in separate experiments each containing one gene of interest to be evaluated. A particular cell line may be generated by co-transfection of a plasmid containing a gene of interest and a recombination site (such as for example, a second Lox, Frt site or attB site) and recombinase/integrase protein or expression plasmid that will catalyze the recombination of the genomic recombination site and the recombination site on the plasmid and therefore the integration of the gene expression plasmid into the genome. All other cell lines may be generated using the same cell clone and the same procedure.

Independent from the method used for targeted integration, the only difference between the cell lines will be the genes of interest that have been integrated into the same genomic locus (loci) and the gene product. Bioproductivity of selected cells may be determined by determining differences in cell performance like viability, cell density, metabolic changes, productivity and quality of the protein. These cell performance characteristics can now be clearly deduced to the gene product. The genes coding for the therapeutic may be either integrated prior to the integration of the cell performance enhancing genes or may be integrated by targeted integration into a different genomic site or the same site as the cell performance enhancing genes e.g. by including it on the same plasmid as the cell performance enhancing gene.

The method is applicable to library screening to identify or validate genes that create a desired cell phenotype or to compare genes from a family to identify the best candidate for downstream cell engineering. Subsequently the identified genes enhancing the same or different phenotypes can be assembled by Multisite Gateway and integrated into the recombination site of the initial host cell line.

Another embodiment is to use targeted integration technology, such as for example, PhiC31, CRE, or FLP and Multisite Gateway to study DNA elements or genes, such as enhancer elements, insulators, chaperones genes, reporters, targets, or secretion leaders, at a specific locus in CHO cells as well as in human lines for bioproduction and drug discovery. Multisite Gateway technology is effective for cloning multiple DNA fragments into one vector without using restriction enzymes. This system can clone 1, 2, 3, 4, 5 or more DNA elements into a single vector. Multisite Gateway allows for combinations of different promoters, DNA elements, and genes to be studied in the same plasmid and targeted at a specific locus using targeted integration system. Instead of transfecting multiple plasmids that can integrate at different loci, the single plasmid carrying different DNA elements can be studied at the same locus and genomic background.

Multisite Gateway may be used to assemble a cassette containing insulator elements, secretion leaders, selection markers, chaperones, novel promoters, att sites, and membrane proteins to generate a retargetable CHO or human lines. DNA elements or genes of interests can be targeted at the specific locus using the R4 integrase, CRE or FLP integrase. For example, a plasmid carrying the PhiC31 and a R4 att site with two different antibiotic selectable markers is transfected in CHO or human cells along with the PhiC31 integrase to generate a stable cell line. An individual clone with the plasmid integrated at the PhiC31 pseudo att site may be isolated and plasmid rescued may be performed to identify the site of integration. Once a stable clone has been obtained, a second plasmid carrying gene of interest, a promoter to drive the expression of the second antibiotic selectable marker, and a R4 attB site that allows for integration at the R4 attP site in the genome, may be transfected along with a R4 integrase expression plasmid into the retargetable clone and select with the second antibiotic selectable marker. Once a stable pool is obtained, individual clones may be isolated and verified for retargeting by PCR or plasmid rescued to confirm the gene of interest has been retargeted at the specified locus in the genome. This platform allows genes or DNA elements to be studied in the same genomic backgrounds and to identify genes or DNA elements that affect bioproduction in CHO cells, as well as generating reporters or targets in human lines for drug discovery.

The following examples are intended to illustrate but not limit the invention.

### Example 1

This example illustrates the site-specific integration of phage integration sites into a human embryonic stem cell line. The human embryonic stem cell line BGO1v (Zeng X et al., Stem Cells 22:292-312 (2004)) was used for these experiments. A plasmid containing the wildtype R4-attP site and the plasmid pcmv-c31Int (encoding the phiC31 integrase) were transfected into the BGO1v cells. Clones were isolated and the genomic integration site determined by sequencing the junction between the plasmid and genomic DNA. The results of this analysis are shown in Table 1. Out of a total of 32 BGO1v clones for which reliable integration data have been determined, 5 were a result of random integration (not shown), and 2 were a mix of site-specific integration and random integration. Three other clones showed integration into multiple pseudo attP sites. Integration into multiple pseudo attP sites may be the result of integration into multiple sites within one cell or multiple cells with different integration events. Of the remaining 23 clones, 18 clones showed integration into 6 pseudo sites, with the most favored pseudo site being located at Chromosome 13q32.3. Further, two pseudo sites identified in this preliminary study (20q11.22 and 21q21.1) have been previously identified and found to be transcriptionally active in terminally differentiated tissue culture lines (HEK 293 and HEPG2).

TABLE 1

| No. of | Cell | | |
|---|---|---|---|
| Clones | Type | Plasmid | Genomic Location |
| 6 | BG01v | R4-attP | 13q32.3 |
| 4 | BG01v | 1 hOG, 3 R4-attP | 6p12.1 |
| 2 | BG01v | R4-attP | 2q35 |
| 2 | BG01v | R4-attP | 10p12.31 |
| 2 | BG01v | hOG | 17q23.3 |
| 2 | BG01v | R4-attP | 21q21.1 |
| 1 | BG01v | hOG | 20q11.22 |
| 1 | BG01v | hOG | 7q33 |
| 1 | BG01v | hOG | 9p24.2 |
| 1 | BG01v | R4-attP | 12q21.2 |
| 1 | BG01v | hOG | 17p11.2 |
| 1 | BG01v | hOG | 11q23.3, 17q23.3, 9q21.13 |
| 1 | BG01v | R4-attP | 9q31.2, 11q24.2 |
| 1 | BG01v | hOG | 6p25.2, 13q13.3 |
| 1 | BG01v | R4-attP | 5q32, random integration |
| 1 | BG01v | R4-attP | 13q32.3, random integration |

### Example 2

This example illustrates the development of an embryonic stem cell line expressing a protein under the control of a developmentally regulated transcription factor. The transcription factor chosen for these experiments is Oct-4 Oct-4 is a transcription factor that is coded for by the Pou5f1 gene. Oct-4 is thought to influence several genes expressed during early embryonic development, and thus, may be very important to the processes of development and cell differentiation. Oct-4 null embryos develop to the blastocyst stage but fail after implantation. These data suggest that Oct-4 plays a central role during cell differentiation in developing embryos.

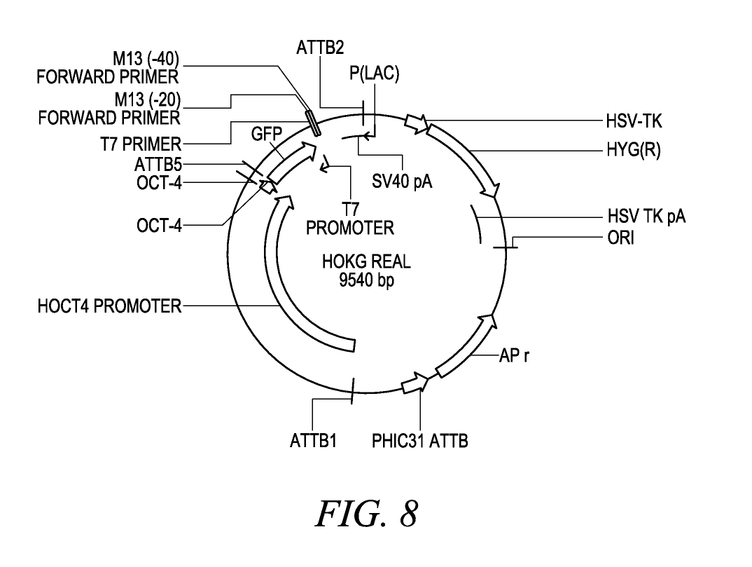

The plasmid used to create the ID1 Oct-4 GFP cell line was hOKG Real and is shown in FIG. 8. The plasmid was constructed using the methods of the invention described above. The destination vector was plasmid pB2H1R1R2DEST1 and the entry vectors were L1-hFLOct4Pr-R5 and L5-kGEPSVpA-L2. An LR cloning reaction using LR Clonase II (Invitrogen Catalog #11791-100) was incubated for 16 hours at 16° C. The LR reaction was then transformed into TOP10 E. coli and plated on LB-agar with Ampicillin A large-scale preparation of plasmid DNA was made using the PureLink HiPure Plasmid Maxiprep kit (Invitrogen catalog #K2100-07).

The hOKG and pcmv-c31Int (phiC31 integrase) plasmids were transfected into BGO1v cells using lipofectamine. Four days after transfection, drug selection with Hygromycin was begun to select for cells expressing the transfected Hygromycin resistance gene under the control of the Oct-4 promoter. Subcloning was begun 7 days after transfection and a second round of drug selection conducted on the isolated clones one month after transfection. Stable clones were established approximately 6 weeks after transfection.

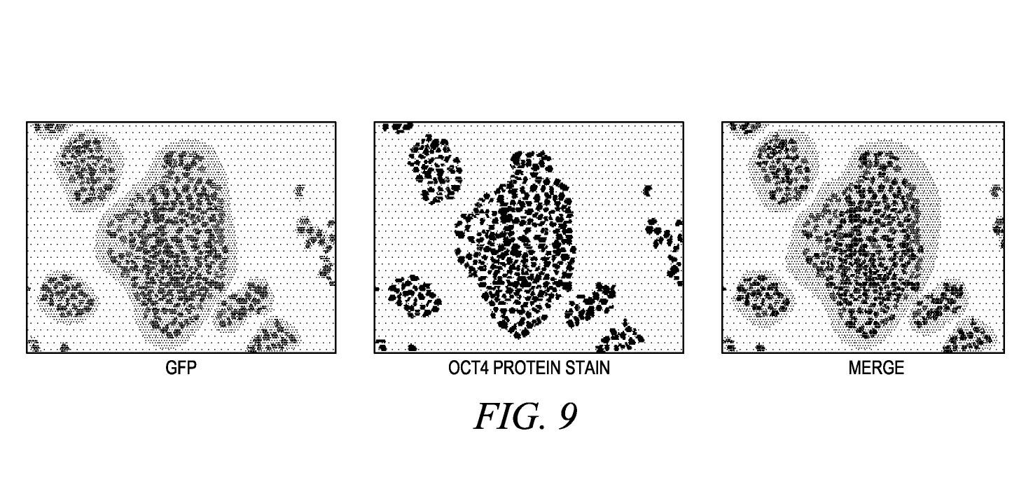

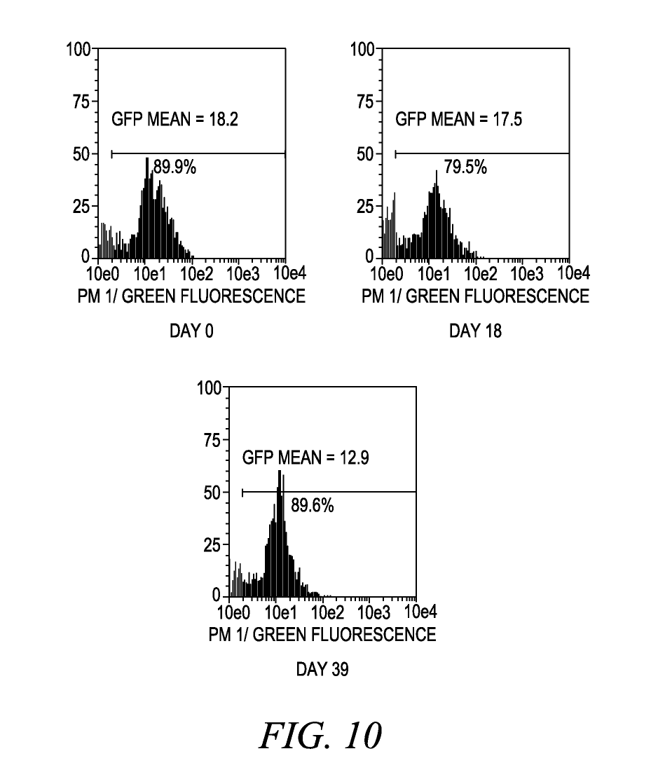

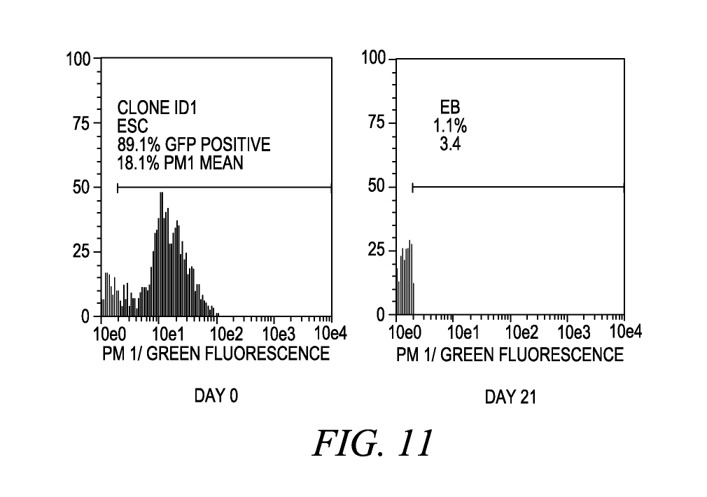

FIG. 9 shows the combined expression of Green Fluorescent Protein (GFP) and Oct-4 protein in the cloned cells. Expression of GFP was stable for at least 39 days as shown in FIG. 10. When BGOv1 cells are allowed to differentiate, the activity of the Oct-4 promoter, which is only functional during early embryonic development, is down regulated. This characteristic is maintained in the engineered Oct-4-GFP BGO1v cells as shown in FIG. 11. When the BGO1v cells were allowed to differentiate for 21 days, the expression of GFP under the control of the Oct-4 promoter was lost. This demonstrates that embryonic stem cells engineered using methods of the present invention retain their biological properties and can serve as model systems for early embryonic development and differentiation.

### Example 3

This example illustrates the use of phiC31 integrase to create variant human embryonic stem cell (hESC)-derived lines containing the GFP gene driven by either the human Oct4 promoter or the human EF1α promoter. We also describe a simplified vector construction design using a targeting vector that is a substrate for Multisite Gateway™. This greatly reduces the effort involved in cloning, and allows one to create multiple constructs in the same background and with little effort. The combination of Multisite Gateway technology and site-specific recombinases provides a powerful tool for the construction of transgenic lines in human embryonic stem cells, which in turn can be used as versatile platforms for the study of stem cell biology.

### Plasmid Construction

The plasmids used in this study are shown in. The plasmid pCMV-phiC31 Int has been described earlier (Groth et al. Proc Natl Acad Sci USA. 97:5995-6000 (2000)). The plasmid pB2H1-DEST was cloned as follows. The phiC31 attB site was amplified from the plasmid pBC-PB and cloned into pCR2.1™ using the TA Cloning Kit (Invitrogen Corporation, Carlsbad, Calif.) to generate pCR2.1-phiC31attB. This plasmid was restricted with EcoRI to release the attB fragment, and treated with Klenow to generate blunt ends. This fragment was ligated with ZraI-restricted pUC19 vector to generate pUC-phiC31attB2. An expression cassette containing the Hygromycin phosphatase gene driven by the HSV-TK promoter was amplified from pTKHyg and T/A cloned into pCR2.1™. The resulting plasmid was restricted with SpeI and EcoRV, treated with Klenow to generate blunt ends, and ligated with pUC-phiC3 lattB2 restricted with AflIII and treated with Klenow to generate pB2H1. A fragment containing the R1-R2DEST cassette was amplified from pUC-DEST (Invitrogen Corporation) and T/A cloned into pCR2.1TM. The resulting plasmid was restricted with SpeI and EcoRV, treated with Klenow, and cloned into pB2H1 treated with SalI and Klenow to generate the plasmid pB2H1-R1R2DEST. This plasmid was used as a recipient for the expression constructs used in this study.

A 3.2 kb fragment containing the human Oct4 promoter (Nordhoff et al. Mamm Genome 12:309-317 (2001), Yeom et al. Development 122:881-894 (1996)) was amplified from human genomic DNA using the primers hO-For (5′-GGAGAGGTGGGCCTCACC-3′) (SEQ ID NO:4) and hO-Rev (5′-GGGGAAGGAAGGCGCCCC-3′) (SEQ ID NO:5). The resulting fragment was TA cloned into pCR2.1™ to generate pCR2.1-phOct4. Assembly of the final phOct4-GFP and pEF1a-GFP expression constructs was accomplished by using protocols recommended for MULTISITE GATEWAYT™.

Cell Culture and Transfection

BG01v cells (49, XXY, +12, +17,) were obtained from BresaGen, Inc. SA002 cells (47, +13, XY) were obtained from Cellartis AB (Goteborg, Sweden). All reagents were obtained from Invitrogen Corporation (Carlsbad, Calif., USA) unless indicated otherwise. The cells were maintained either on a mouse embryonic fibroblast (MEF) feeder layer in DMEM/F12 medium supplemented with 20% KSR, 4 ng/ml of bFGF, 1 ml of non-essential amino acids, and 100 μM β-mercapto ethanol or on Matrigel (BD Biosciences, New Jersey, USA) in the same medium conditioned on MEF feeder layer. Fresh medium was provided to the cells every day, and the cells were passaged every 4 to 5 days.

One day prior to transfection with Lipofectamine 2000 (Invitrogen Corporation, Carlsbad, Calif.), cells were treated with Accutase (Sigma, St. Louis, Mo., USA) and plated on Matrigel in conditioned medium. Lipofectamine 2000-mediated transfection was carried out according to manufacturer's protocol. We typically used 4 μg of the expression vector and 4 μg of the phiC31 integrase expression vector to transfect 2 million cells. Control transfections omitted the phiC31 integrase plasmid or the GFP expression vector. After transfection, cells were allowed to recover for 1 day, and selection was started with medium containing Hygromycin at a concentration of 10 μg/ml. After 14-21 days of selection, individual colonies were manually picked and expanded for further analysis.

Electroporation was carried out with the ECM630 electroporator (BTX). Six to eight million cells were harvested using Accutase and resuspended in 800 μl of OptiPro™ SFM (Invitrogen Corporation). These cells were placed in an electroporation cuvette with a gap of 0.4 cm. Cells were electroporated with a pulse of 500V at 250 μF. Electroporated cells were plated on MEF feeders and allowed to recover for 48-72 hours before selection was started with hygromycin (10 μg/ml, Invitrogen). As with lipid-mediated transfection, individual drug-resistant clones were manually picked and expanded for further analysis.

Plasmid Rescue and Sequence Analysis

Genomic DNA isolated from individual clones was restricted with the restriction enzymes NheI, SpeI and XbaI. The enzymes were heat-inactivated, and the DNA was self-ligated at low DNA and T4 DNA Ligase concentrations. After overnight incubation at 16° C., the DNA was extracted with phenol:chloroform, ethanol precipitated, and resuspended in water. Electrocompetent DH10B E. coli were then electroporated with the ligated DNA using the Bio-Rad Gene Pulser II (Biorad Corporation, Hercules, Calif.) using recommended conditions. The resulting transformation was plated on LB-agar plates containing ampicillin Plasmid DNA isolated from the resulting colonies was sequenced using the primer ChoSeqR (5′-TCCCGTGCTCACCGTGACCAC-3′) (SEQ ID NO:6). Sequence data were analyzed using Sequencher software. The genomic integration site was determined by matching the sequence read to the database at BLAT (http://genome.ucsc.edu/).

Analysis of 23 pseudo site sequences rescued in this study was carried out by the web-based MEME motif finder (http://meme.sdsc.edu/meme/meme.html). This program was utilized to find motifs ranging from 6-50 base pairs in 100 base pairs of sequence surrounding the point of cross-over. The wild-type phiC31 attP site was also included in the analysis. A common motif was discovered in all the pseudo sites, and a consensus sequence was generated based on these analyses using WebLogo Version 2.8.2 (http://weblogo.berkeley.edu/).

Differentiation and Silencing Assays

Cells were induced to form embryoid bodies in differentiation medium as described with some modifications. Differentiation medium is composed of DMEM/F12 supplemented with 10% FBS, 1% NEAA, 100 μM β-mercaptoethanol. Four days after the start of differentiation, embryoid bodies were plated on culture plate to be differentiated further as monolayers. After 14 days, the differentiation potential was measured by immunocytochemistry for markers specific for the three different lineages. Primary antibodies were obtained from various sources and used at the following dilutions: Pluripotent marker of Oct4 (1:500, Abcam), Endoderm marker of Alpha-Fetoprotein (1:500, Santa Cruz), Mesoderm marker of Smooth Muscle Actin (1:200, Sigma) and Brachyury (1:1000, R&D Systems), Ectoderm marker of Beta III Tubulin (TUJ1) (1:1000, Invitrogen) and Nestin (1:500, BD Biosciences). Secondary markers were obtained from Molecular Probes (Eugene, Oreg.) and used at the following dilutions: Alexa 594 conjugated anti-mouse IgG (1:1000) and Alexa 594 conjugated anti-rabbit IgG (1:1000).

Plasmid Construction and Site-Specific Integration Strategy

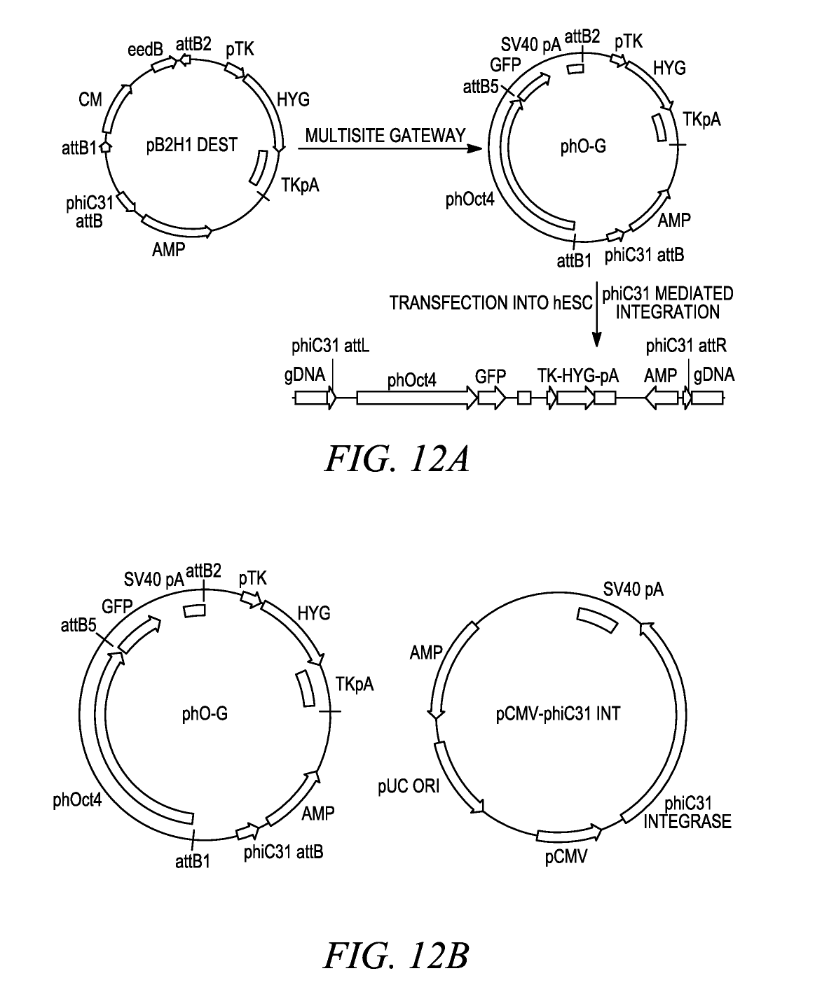

Cloning of recombinant DNA molecules involves multiple steps that can be time-consuming, and in some cases extremely difficult to achieve. To streamline the process of cloning complex expression constructs, we used MULTISITE GATEWAY™ technology. This involved construction of a Destination vector (pB2H1-DEST in our case, FIG. 12, Panel A) which acted as a recipient for the expression elements. Entry vectors containing the promoter and gene to be expressed were constructed via PCR amplification using specific primers flanked by λ phage recombination site sequences. Recombination of the amplified products with the recipient pDONR vectors generated the Entry vectors which could then be used for multiple constructions. Appropriate entry vectors were recombined with the Destination vector in one step to generate expression vectors containing the gene of interest driven by promoter of choice. In this study, we used this strategy to generate two vectors that consist of the GFP gene driven by either the constitutive EF1α promoter, or the hESC-specific human Oct4 promoter (FIG. 12b).

We then used phiC31 integrase to insert the plasmids into the hESC genome. This enzyme directs integration of expression vectors into pseudo attP sites in the human genome in an efficient manner. To this end, we engineered our Destination vector such that it would contain a recombination site for phiC31 integrase. To allow for selection of integration events, we also incorporated the hygromycin phosphotransferase gene driven by the HSV-TK promoter. To obtain cells with integration events, the cells of interest were transfected with the expression vectors along with a plasmid encoding the expression of phiC31 integrase. The integrase protein catalyzed the integration of the expression vector into genomic pseudo attP locations. Stable integration events were selected by expression of the drug-resistance marker present on the plasmids.

The expression constructs were transfected in the absence and presence of the phiC31 integrase plasmid into BG01 v cells. Typically, the frequency of integration after two weeks of drug selection in the presence of integrase was ˜2×10⁻⁵. Data from three controlled experiments show that the average increase in colony number was 1.4-fold over random integration. In the absence of integrase, 80 colonies were obtained from three experiments, and in the presence of integrase, 114 colonies were obtained. These data suggest that phiC31 integrase can mediate integration into pseudo sites in hESC.

Pseudo Site Profile in hESC

To show that clones obtained were the result of phiC31 mediated site-specific integration, the site of integration was determined by a plasmid rescue strategy. The attB-genome junctions were sequenced, and the data analyzed by comparison with the BLAT database (http://genome.ucsc.edu/cgi-bin/hgBlat). Table 2 shows the sites of integration of various clones derived from BG01v or SA002. Out of 90 clones screened, plasmid rescue data were obtained for 56 clones. Of these, 51 clones were a result of phiC31-mediated integration and 5 were a result of random integration. The chromosomal loci for the random integration events were not determined. The 51 integrase-mediated clones showed integration into 23 different pseudo attP sites. As has previously been observed, there were small deletions (5 to 25 bases) observed at the site of integration 11, 15.

TABLE 2
phiC31 pseudo attP sites in hESC

| | | | | | Gene annotation | | |
|---|---|---|---|---|---|---|---|
| Genomic location | Strand | # of clones | Cells | Repeat | Location | Nearest/Upstream gene* | Downstream gene |
| 1p32.3 | − | 1 | BG01v | No | Exon | CDCP2 | |
| 2q35^{a} | + | 2 | BG01v | Yes, AluY | Intergenic | FN1 | DSU |
| 5q32 | − | 1 | BG01v | Yes, HERVH | Intron | SPINK1.eAug05, Intron 1 | |
| 6p11.2^{a} | + | 5 | BG01v | No | Intron | PRIM2A, Intron 5 | |
| 6p25.2 | + | 1 | BG01v | No | Intergenic | SERPINB6 | DKFZp686I15217 |
| 7q33 | + | 1 | BG01v | No | Intron | AKR1B10, Intron 4 | |
| 9p24.2 | + | 1 | BG01v | No | Intergenic | KIAA0020 isoform 1 | tyrorby.aAug05 |
| 9q21.13 | + | 1 | BG01v | Yes, MLT1I | Intron | TRPM3, Intron 1 | |
| 9q31.2^{a} | + | 3 | Both | No | Intron | slulo.bAug05, Intron 1 | |
| 10p12.31 | − | 2 | BG01v | No | Intergenic | danerby.aAug05 | boyloy.aAug05 |
| 10p12.33 | + | 1 | BG01v | No | Intron | CACNB2, Intron 2 | |
| 11q23.3 | + | 1 | BG01v | No | Intron | DSCAML1, Intron 3 | |
| 11q24.2 | + | 1 | BG01v | Yes, MER44A | Intergenic | OR8B8 | smarlorby.aAug05 |
| 12q21.2 | − | 1 | BG01v | No | Exon | lorchar.aAug05, Exon 1 | |
| 12q22 | + | 1 | BG01v | No | Intergenic | SOCS2 | CRADD |
| 13q13.3 | − | 1 | BG01v | No | Intron | TRPC4, Intron 1 | |
| 13q32.3^{a} | +/− | 17 | Both | No | Intron | CLYBL, Intron 2 | |
| 17p11.2 | + | 1 | BG01v | Yes, MIRb | Intron | LRRC48, Intron 4 | |
| 17q23.3^{a} | +/− | 4 | BG01v | No | Intergenic | TLK2 | MRC2 |
| 20q11.22 | + | 1 | BG01v | No | Intron | RALY, Intron 2 | |
| 20q13.32 | + | 1 | SA002 | No | Intron | STX16, Intron 5 | |
| 21q21.1^{a} | − | 2 | BG01v | Yes, HERVLA2 | Intergenic | NRIP1 | USP25 |
| Xq23 | − | 1 | SA002 | No | Intron | ZCCHC16, Intron 2 | |

*If the pseudosite is in an intron, the gene mentioned in this column is that gene. If the pseudo site is intergenic, the gene mentioned is upstream of the pseudo site.
^{a}These pseudo sites were detected in multiple clones, and are considered hotspots for recombination

Our data show that there are numerous hotspots of integration in stem cells, many of which have not been previously reported in other cell types. There are, however, some integration sites that are common to hESC and differentiated cell types like 293, HepG2, and D407 lines. The number of integration events at each pseudo site is shown in Table 2. As shown in a previous study, most of these hotspots are present in introns of genes, with a few present in inter-genic regions or exons 10. In this study, we found that the most commonly used integration sites were present on chromosome 13, chromosome 6, chromosome 21, chromosome 9, chromosome 17 and chromosome 2. Of these, only the site on chromosome 21 has been observed previously. The other hotspots have not been reported in differentiated cell types and seem to be exclusive to hESC. However, the integration sites on chromosome 1, chromosome 6 (6p25.2), and the two sites on chromosome 20 have been reported earlier, suggesting that they might also be hotspots for integration. Since the majority of the clones we analyzed were derived from the BG01v line, we could not make a meaningful comparison as to the pseudo site profile in these cells vis a vis SA002 cells. However, two of the hotspots were present in both cell lines, suggesting that there is at least some commonality between the two independently derived lines. A few clones showed integration into multiple sites (data not shown). It was not clear if the clones that showed integration into multiple sites were a mix of two independent clones or if that clone truly had multiple integrations.

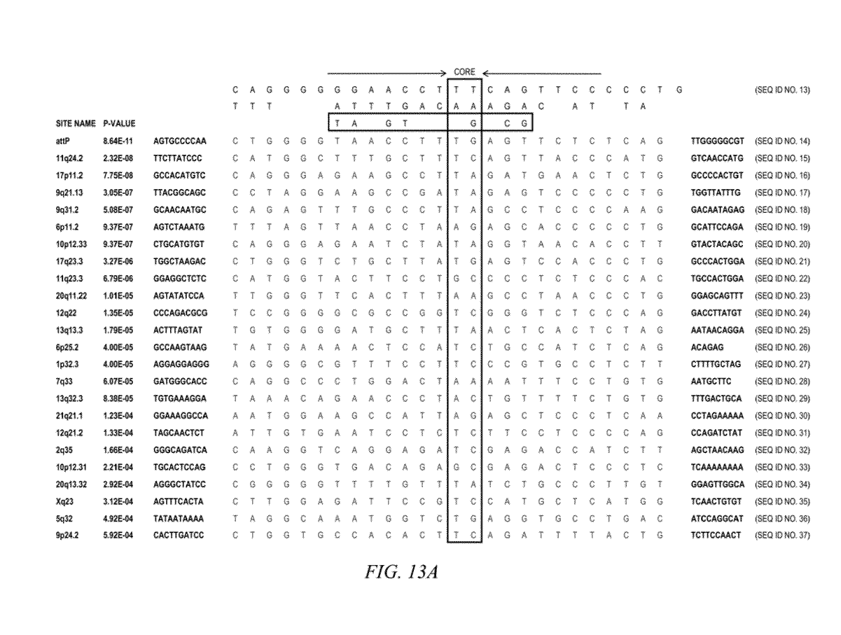

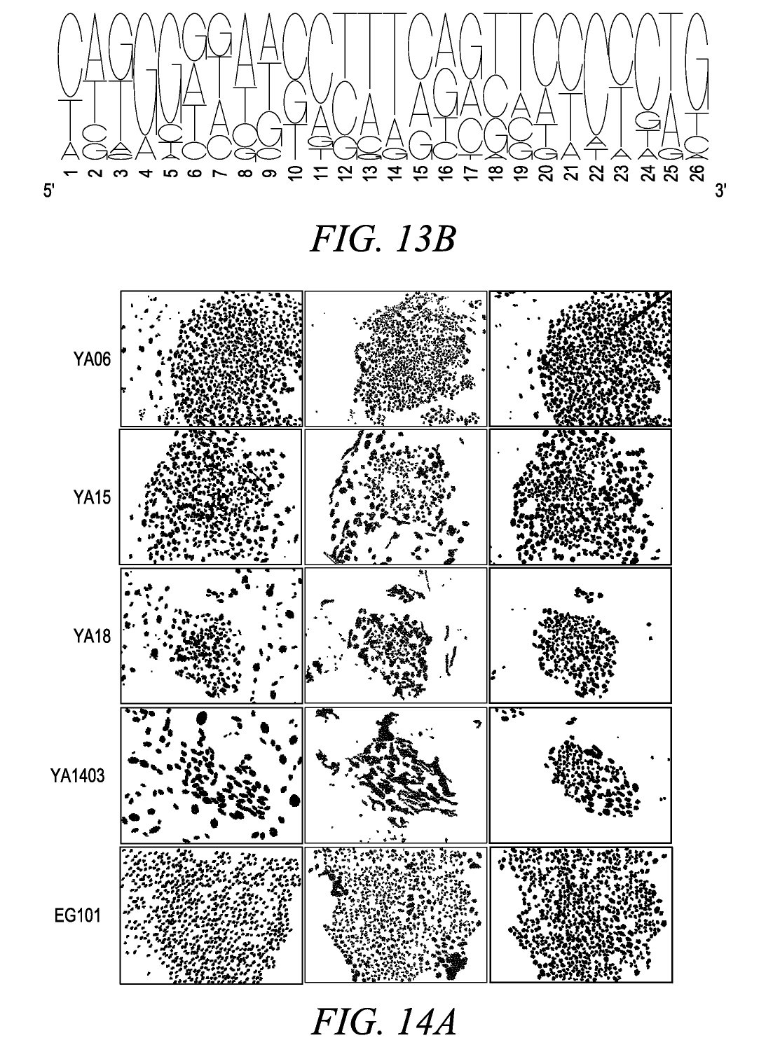

It has previously been reported that pseudo attP sites show some similarity to the native phiC31 attP site, and that they share a common motif that contains a strong inverted repeat (Chalberg et al. J Mol Biol 357:28-48 (2006)). The pseudo sites observed in hESC were subjected to similar analysis, and we found that these sites shared a common motif with the phiC31 attP site (FIG. 13A). This motif is present close to the crossover region in most of the sites, suggesting involvement in the recombination reaction. A consensus sequence for this motif was derived using the MEME motif finder (Bailey et al. Proceedings International Conference on Intelligent Systems for Molecular Biology ISMB. 2:28-36 (1994)). The consensus sequence of this motif is shown in FIG. 13A. The consensus shows a strong inverted repeat centered on the core, providing further evidence to the hypothesis that the integrase binds to each half-site (Smith et al. Mol Microbiol. 44:299-307 (2002)). A sequence logo diagram of the consensus sequence is shown in panel B of FIG. 13.

Generation of GFP-Expressing hESC Lines

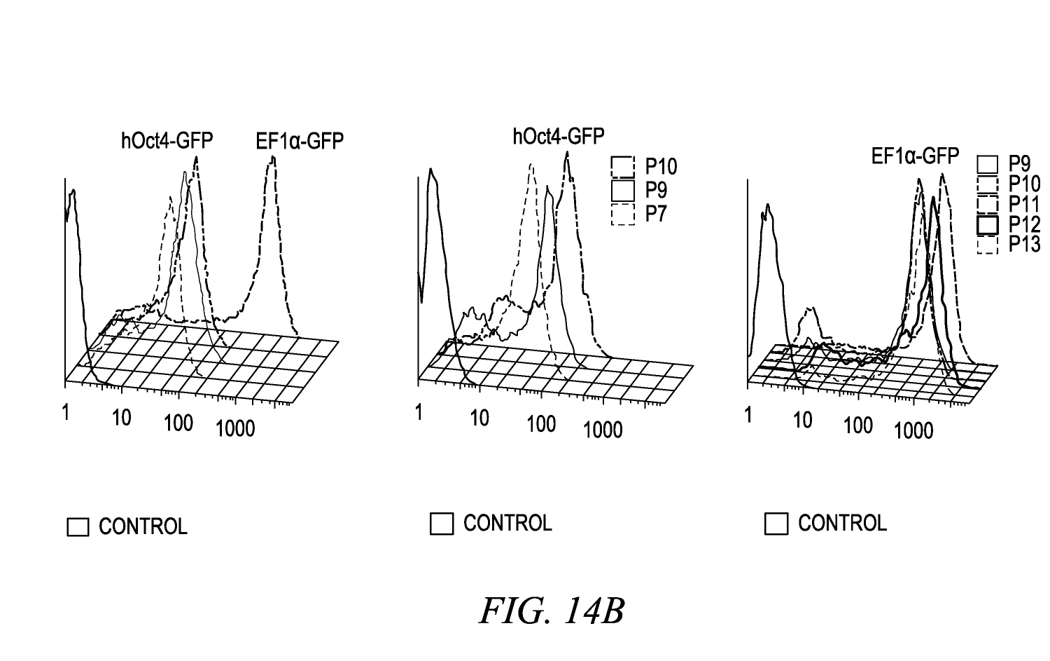

We evaluated both lipid-mediated transfection as well as electroporation to introduce DNA into BG01v and SA002 cells. Typically, we obtained transfection efficiencies ranging from 5-20% with minimal cell death. After transfection, the cells were allowed to recover and then placed under selection with Hygromycin. Drug-resistant colonies obtained after two weeks of selection were picked and expanded for further observation. Multiple GFP-expressing clones were obtained with both cell types, and the colonies that were closest morphologically to the parent lines were selected for further analysis. FIG. 14A shows bright-field and fluorescent microscope views of three different BG01v-derived lines and one SA002-derived line. Counter-staining with an antibody specific for human Oct4 demonstrates that Oct4 and GFP expression are co-localized.

A similar strategy was employed to obtain BG01v-derived lines expressing the GFP gene driven by the constitutive human EF1α promoter 34. As shown in FIG. 14A, the EF1α promoter directs strong expression of GFP in these cells. FACS analysis of three independent Oct4-GFP clones and one EF1α-GFP clone reveal that the EF1α promoter directs higher levels of expression compared to the hOct4 promoter (FIG. 14B, Panel I). This expression is maintained upon long-term culture, as shown in FIG. 14B, Panels II and III. Irrespective of the promoter, there is no significant reduction in GFP expression even after 10 passages, which is approximately 4 to 5 weeks in culture.

Characteristics of GFP Lines

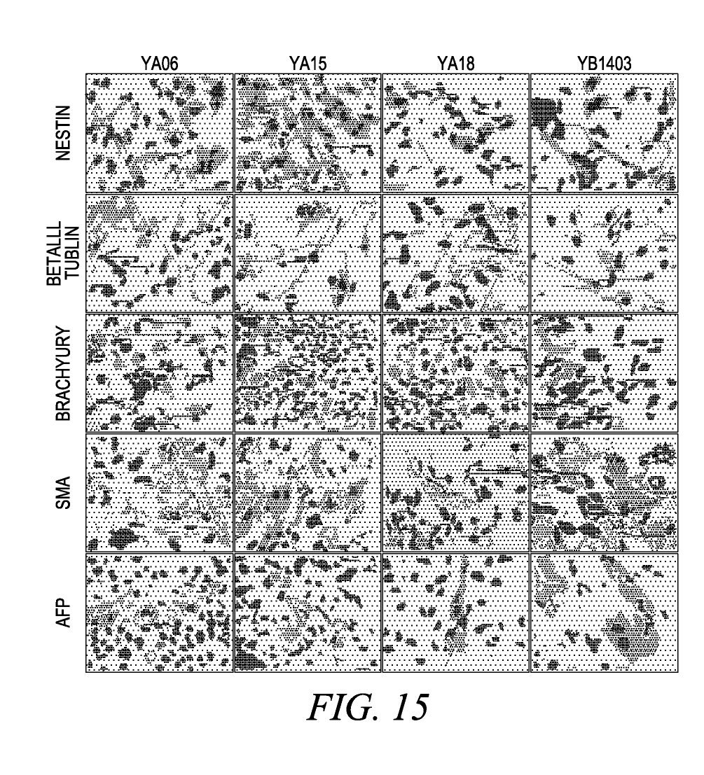

Three independent BG01v-phOct4-GFP clones (YA06, YA15 and YA18) and one SA002-phOct4-GFP clone (YB1403) were studied for their ability to differentiate into the three germ layers by inducing formation of embryoid bodies (EBs). Immunostaining of the embryoid bodies are shown in FIG. 15. Expression of endodermal (α-Fetoprotein), ectodermal (βIII-Tubulin and Nestin) and mesodermal (muscle specific actin and brachyury) markers was detected in EBs derived from all four lines.

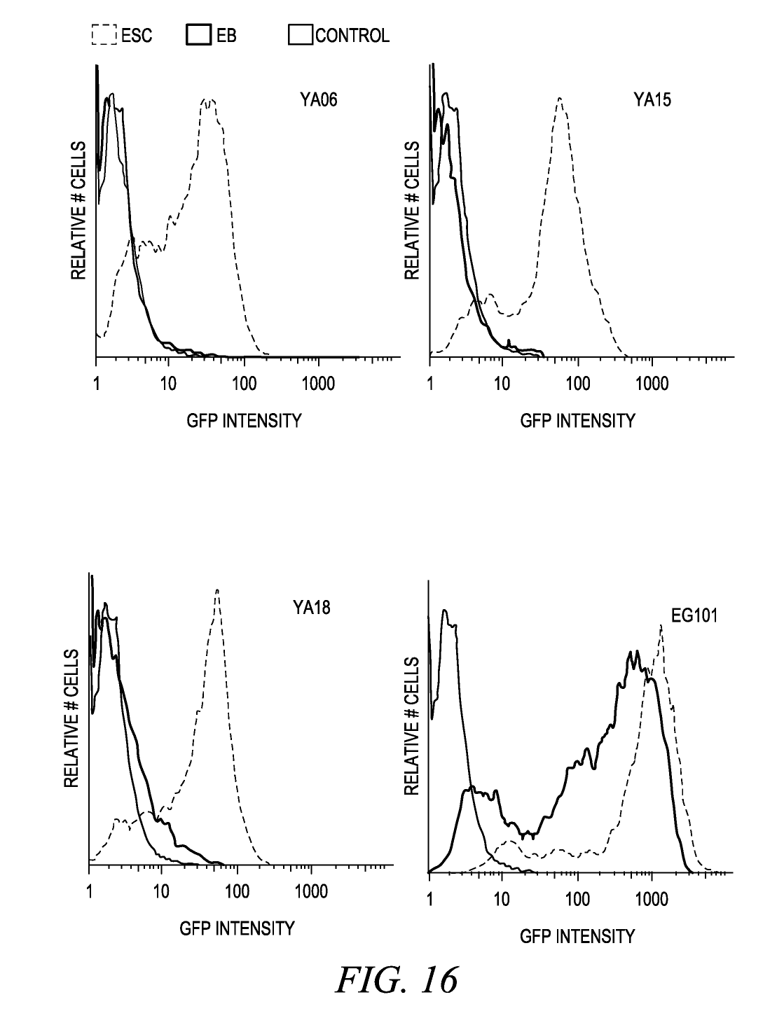

Differentiation of human ESC results in down-regulation of Oct4 expression. To demonstrate that the promoter fragment used in this study was subject to the same regulation as the native Oct4 promoter, expression of GFP was monitored in EBs derived from the Oct4-GFP transgenic lines. As expected, expression of GFP driven by the human Oct4 promoter was eliminated following differentiation (FIG. 16), showing that elements required for proper control of gene expression are present in the promoter fragment. Further, upon knockdown of Oct4 protein message with RNAi, we noticed a significant decrease in GFP fluorescence (data not shown). In contrast, expression of GFP driven by the EF1α promoter was still present upon differentiation.

### Example 4

This example illustrates the integration of genes of interest into a specific locus and their effect on cell bioproduction.

Generation of DNA Vectors

PCR primers are designed according to DNA sequences to include promoters, gene of interest, or DNA elements such as enhancers, insulators, or IRES elements. Primers are designed with appropriate flanking recombination Att sequences (see Multisite Gateway Pro manual (Catalog #12537-100)) to allow PCR fragments to be cloned into appropriate entry vectors. Once entry vectors are obtained, the final expression constructs are assembled using different entry vectors to obtain the desired configuration. (see Multisite Gateway Manual).

Generation Retargeting Cell Lines in CHOS

DNA construct containing the retargeting Att site is transfected into CHOS. 38 ug of DNA is incubated with 38 ul of Freestyle Max and incubated at RT for 10 min in serum free medium. The mixture is added to 3×10E7 CHOS cells in 30 ml of CD CHO medium, and incubated overnight in the shaker at 37C. Next day, medium is replaced with fresh CD-CHO medium. After 48 hours post transfection, antibiotic is added to medium and cells are replaced with fresh medium containing antibiotic every other day. After 14 days, stable pool of CHOS containing the retargeting Att site is obtained. The pool can be subcloned and expanded to obtain a clone containing the retargeting Att site.

Generation of HEK 293 Retargeting Cell Line

DNA construct containing the retargeting Att site is transfected into HEK 293 cells. Cells are plated onto 6 well plate the day before transfection to obtain approximately 70% confluent. 1.6 ug of DNA is added to 4 ul of Lipofectamine-2000 in 100 ul of OptiMEM medium. The mixture is incubated for 15 min at room temperature and added to one of the 6 well plate and incubated for 48 hrs. After 48 hours, medium is replaced with medium containing antibiotic. Cells are replaced with new medium containing antibiotic every other day until single colonies arise.

Retarget Gene of Interest into Specific Locus

Lipofectamine-mediated transfection in HEK293 Retargeting Line is conducted as follows.
i. About 90% confluent HEK293 retargeting cell line is washed once with PBS(−/−) and 1 ml of TrypLE is applied.
ii. After 2 mins, 1 ml of medium is added and gently pipetted to resuspend cells using a 5 ml serological pipette. Harsh triturating is avoided to make single cell suspensions.
iii. Cells are transferred to 15 ml conical tubes medium is added up to 5 ml.
iv. Cells are spun at 1000 rpm for 2 mins at room temperature
v. Medium is aspirated and cells are replated in a 6 well plate 24 hours prior to transfection to obtain approximately 70% confluency next day.
vi. On the day of transfection, 100 ul of Opti-MEM® I Reduced Serum Medium without serum is aliquoted into a 1.5 ml microcentrifuge tube, DNA (1.6 ug total: 0.8 ug of gene of interest and 0.8 ug of integrase) is added and mixed gently by pipetting up and down twice using a 1 ml pipette. Optimize DNA amount if required.
vii. A tube of Lipofectamine™ 2000 is mixed gently, and then diluted at 4.0 ul in 100 μl of Opti-MEM® I Medium.
viii. Incubation is conducted for 5 minutes at room temperature. After the 5 minute incubation, 100 ul of DNA mixture is combined with 100 ul Lipofectamine mixture. Mixing is done gently and incubation is conducted for 20 minutes at room temperature (solution may appear cloudy). Note: Complexes are stable for 6 hours at room temperature.
ix. 200 μl of complexes are added to dishes containing cells and 2 ml of fresh medium. Plates are mixed gently by rocking the plate back and forth.
x. Cells are replaced with medium with antibiotic every other day until single colonies arise. Colonies can be pooled together or isolate single clone and expand.

TRPM8 and CCKAR genes were transfected in HEK294 retargeting cell line. A pool of each gene was obtained and subjected for GPCRs agonist-stimulated and antagonist-inhibited calcium signaling assays. Results of those assays are set forth in FIGS. 20 and 21.

### FreeStyle MAX-Mediated Transfection in CHOS

DNA construct containing the gene of interest is transfected into CHOS. 38 ug of DNA (17.5 ug of gene of interest and 17.5 ug of integrase) is incubated with 38 ul of Freestyle Max and incubated at RT for 10 min in serum free medium. The mixture is added to 3×10⁷ CHOS cells in 30 ml of CD CHO medium, and incubated overnight in the shaker at 37° C. Next day, cells are replaced with fresh CD-CHO medium for 48 hours. After 48 hours post transfection, antibiotic is added to medium and replaced with fresh medium containing antibiotic every other day. After 14 days, a stable pool of CHOS containing the gene of interest is obtained. The pool can be directly screened for protein expression or subcloned into single clone.

GFP gene was retargeted into CHOS retargeting line and a stable pool was obtained. GFP fluorescent can be visualized as illustrated in FIG. 22.

All publications, U.S. Patents, U.S. Patent Applications and non-U.S. patent documents cited herein are hereby incorporated by reference in their entirety. Although the invention has been described with reference to the above examples, it will be understood that modifications and variations are encompassed within the spirit and scope of the invention. Accordingly, the invention is limited only by the following claims.

What is claimed is:

1. A method for generating a cell which contains genetic material inserted into the cellular genome, the method comprising:
a) transfecting a population of cells with a first nucleic acid molecule, the nucleic acid molecule further comprising a first recombination site, a first selectable marker and a second selectable marker;
b) selecting cells from the population in which the first nucleic acid has been integrated into the genome;
c) transfecting the cells selected by use of the first selectable marker with a second nucleic acid comprising at least one genetic element for expression in the cell, a promoter and a second recombination site and providing to the selected cells a recombinase specific for the first and second recombination sites such that the second nucleic acid is inserted into the genome of the cell by site-specific recombination; and
d) selecting cells in which the second nucleic acid has been integrated into the genome.

2. The method of claim 1, wherein the cells are selected from the population in which the first nucleic acid has been integrated into the genome by use of the first selectable marker.

3. The method of claim 1, wherein selecting cells in which the second nucleic acid has been integrated into the genome is by use of the second selectable marker.

4. The method of claim 1, wherein the first nucleic acid further comprises a third recombination site.

5. The method of claim 4, wherein the third recombination site is complimentary to a pseudo recombination site present in the cell and wherein a recombinase specific for the third recombination site and the pseudo recombination site is provided to the cell such that the plasmid is inserted into the genome of the cell by site-specific recombination.

6. The method of claim 1, wherein the first nucleic acid is integrated into the genome of the cell by homologous recombination.

7. The method of claim 1, wherein the first recombination site is a wild type R4 integration site.

8. The method of claim 1, wherein the first recombination site is a wild type phiC31 integration site.

9. The method of claim 1, wherein the promoter in the second nucleic acid is positioned such that upon completion of the recombination reaction, the promoter is operably linked to the second selectable marker.

10-13. (canceled)

14. A method for identifying a genomic locus suitable for expressing a heterologous nucleic acid molecule wherein the genomic locus is not essential for cellular function and wherein the genomic locus remains transcriptionaly active during cellular differentiation, the method comprising:
a) transfecting the cell with a first nucleic acid, said first nucleic acid further comprising a first recombination site, a first selectable marker and a second selectable marker;
b) selecting cells in which the first nucleic acid has been integrated into the genome by use of the first selectable marker;
c) transfecting the cells selected by use of the first selectable marker with a second nucleic acid comprising a promoter and a second recombination site and providing to the selected cells a recombinase specific for the first and second recombination sites such that the second nucleic acid is inserted into the genome of the cell by site-specific recombination;
d) selecting cells in which the second nucleic acid has been integrated into the genome by use of the second conditional selectable marker;
e) mapping the genomic location of the integrated second nucleic acid;
f) differentiating the cells selected with the second selectable marker to each of ectoderm, endoderm and mesoderm cell types in the presence of the selection agent for the second selectable marker; and
g) identifying the mapped genomic locus of the cells which are able to differentiate to each of ectoderm, endoderm and mesoderm cell types in the presence of the selection agent for the second selectable marker.

15. The method of claim 14, wherein the first nucleic acid further comprises a third recombination site.

16-20. (canceled)

21. A method for directly isolating cells expressing a transfected nucleic acid molecule comprising:
a) transfecting an embryonic stem cell with a first nucleic acid, such that the first nucleic acid integrates into a pseudo recombination site known to be located in a genomic locus that is not essential for cellular function and wherein the genomic locus remains transcriptionaly active during cellular differentiation, wherein the first nucleic acid further comprising a first recombination site complimentary to the pseudo recombination site, a first selectable marker and a second conditional selectable marker;
b) selecting embryonic stem cells in which the first nucleic acid has been integrated into the genome by use of the first selectable marker;
c) creating a transgenic animal derived from the transfected embryonic stem cell;
d) constructing a second nucleic acid comprising a promoter and a second recombination site;
d) isolating cells from the transgenic mouse and transfecting them with the second nucleic acid and providing to the cells a recombinase specific for the first and second recombination sites such that the second nucleic acid is inserted into the genome of the cell by site-specific recombination; and
e) directly isolating transfected cells which grow in the presence of the selection agent for the second conditional selectable marker.

22. The method of claim 21, wherein the first recombination site is a wild type R4 integration site.

23. The method of claim 21, wherein the first recombination site is a wild type phiC31 integration site.

24. The method of claim 21, wherein the promoter in the second nucleic acid is positioned such that upon completion of the recombination reaction, the promoter is operably linked to the second conditional selectable marker.

25. The method of claim 21, wherein the second nucleic acid further comprises a genetic element for expression in the cell.

26. The method of claim 21, wherein the cells isolated from the transgenic mouse are embryonic stem cells.

27-31. (canceled)

32. The method of claim 25, wherein the cells isolated from the transgenic mouse are adult stem cells.

33-48. (canceled)


SEQUENCE LISTING (38 sequences)

SEQ ID NO: 1 - DNA, 34 bp, Artificial Sequence
  synthetic construct loxP
ataacttcgtatagcatacattatacgaagttat

SEQ ID NO: 2 - DNA, 34 bp, Saccharomyces cerevisiae
gaagttcctatacttctagaagaataggaacttc

SEQ ID NO: 3 - DNA, 12 bp, Artificial Sequence
  synthetic construct resolvase
gaagcagtggta

SEQ ID NO: 4 - DNA, 18 bp, Artificial Sequence
  synthetic construct primer
ggagaggtgggcctcacc

SEQ ID NO: 5 - DNA, 18 bp, Artificial Sequence
  synthetic construct primer
ggggaaggaaggcgcccc

SEQ ID NO: 6 - DNA, 21 bp, Artificial Sequence
  synthetic construct primer
tcccgtgctcaccgtgaccac

SEQ ID NO: 7 - DNA, 21 bp, Artificial Sequence
  synthetically modified attB site
ctgcttttttgtacaaacttg

SEQ ID NO: 8 - DNA, 21 bp, Artificial Sequence
  synthetically modified attB site
cagctttcttgtacaaagttg

SEQ ID NO: 9 - DNA, 21 bp, Artificial Sequence
  synthetically modified attB site
caactttattatacaaagttg

SEQ ID NO: 10 - DNA, 21 bp, Artificial Sequence
  synthetically modified attB site
caacttttctatacaaagttg

SEQ ID NO: 11 - DNA, 21 bp, Artificial Sequence
  synthetically modified attB site
caacttttgtatacaaagttg

SEQ ID NO: 12 - DNA, 21 bp, Artificial Sequence
  synthetically modified attB site
caactttttaatacaaagttg

SEQ ID NO: 13 - DNA, 27 bp, Artificial Sequence
  Artificial Concensus
caggggggaacctttcagttccccctg

SEQ ID NO: 14 - DNA, 46 bp, Homo sapien
agtgccccaactggggtaacctttgagttctctcagttgggggcgt

SEQ ID NO: 15 - DNA, 46 bp, Homo sapien
ttcttatccccatggctttgctttcagttacccatggtcaaccatg

SEQ ID NO: 16 - DNA, 46 bp, Homo sapien
gccacatgtccagggagaagccttagatgaactctggccccactgt

SEQ ID NO: 17 - DNA, 46 bp, Homo sapien
ttacggcagccctaggaagccgatagagtccccctgtggttatttg

SEQ ID NO: 18 - DNA, 46 bp, Homo sapien
gcaacaatgccagagtttgcccttagcctccccaaggacaatagag

SEQ ID NO: 19 - DNA, 46 bp, Homo sapien
agtctaaatgtttagttaacctaagagcaccccctggcattccaga

SEQ ID NO: 20 - DNA, 46 bp, Homo sapien
ctgcatgtgtcagggagaatctataggtaacaccttgtactacagc

SEQ ID NO: 21 - DNA, 46 bp, Homo sapien
tggctaagacctgggtctgcttatgagtccaccctggcccactgga

SEQ ID NO: 22 - DNA, 46 bp, Homo sapien
ggaggctctccatggtacttcctgcccctctcccactgccactgga

SEQ ID NO: 23 - DNA, 46 bp, Homo sapien
agtatatccattgggttcactttaagcctaaccctgggagcagttt

SEQ ID NO: 24 - DNA, 46 bp, Homo sapien
cccagacgcgtccggggcgccggtcgggtctcccaggaccttatgt

SEQ ID NO: 25 - DNA, 46 bp, Homo sapien
actttagtattgtggggatgctttaactcactctagaataacagga

SEQ ID NO: 26 - DNA, 42 bp, Homo sapien
gccaagtaagtatgaaaactccatctgccatctcagacagag

SEQ ID NO: 27 - DNA, 46 bp, Homo sapien
aggaggagggaggggcgtttccttcccgtgcctcttcttttgctag

SEQ ID NO: 28 - DNA, 44 bp, Homo sapien
gatgggcacccaggccctggactaaaatttcctgtgaatgcttc

SEQ ID NO: 29 - DNA, 46 bp, Homo sapien
tgtgaaaggataaacagaaccctactgttttctgtgtttgactgca

SEQ ID NO: 30 - DNA, 46 bp, Homo sapien
ggaaaggccaaatggaagccattagagctccctcaacctagaaaaa

SEQ ID NO: 31 - DNA, 46 bp, Homo sapien
tagcaactctattgtgaatcctctcttcctccccagccagatctat

SEQ ID NO: 32 - DNA, 46 bp, Homo sapien
gggcagatcacaaggtcaggagatcgagaccatcttagctaacaag

SEQ ID NO: 33 - DNA, 46 bp, Homo sapien
tgcactccagcctgggtgacagagcgagactccctctcaaaaaaaa

SEQ ID NO: 34 - DNA, 46 bp, Homo sapien
agggctatcccgggggttttgtttatctgcccttgtggagttggca

SEQ ID NO: 35 - DNA, 46 bp, Homo sapien
agtttcactacttggagattccgtccatgctcatggtcaactgtgt

SEQ ID NO: 36 - DNA, 46 bp, Homo sapien
tataataaaataggcaaatggtctgaggtgcctgacatccaggcat

SEQ ID NO: 37 - DNA, 46 bp, Homo sapien
cacttgatccctggtgccacacttcagattttactgtcttccaact

SEQ ID NO: 38 - DNA, 5 bp, Artificial Sequence
  synthetic construct of resolvase
tcaat